SEQUENCE LISTING (13 sequences)

SEQ ID NO: 1 - protein, 130 aa, Artificial Sequence
  2A7 VH
EFEVKLQQSGGGLVQPGGSRKLSCEASGFTISSFGMHWVRQAPEKGLEWVAYISSGSNTIYYADTVKGRFTISRDIPKNTLFLQMTSLRSEDTAMYYCARRLAYWGQGTLVTVSAAKTTPPSVYPLAPRS

SEQ ID NO: 2 - protein, 119 aa, Artificial Sequence
  2A7 VL
ELDIVLTQTPAIMSASPGEKVTMTCSASSSVSSMYWYQQKPGSSPRLLIFDTSNLASGVPVRFSGSGSGTSYSLTISRMEAEDVATYYCLQWSSYPLTFGAGTKLELKRADAAPTVSAC

SEQ ID NO: 3 - protein, 8 aa, Artificial Sequence
  2A7 VH CDR1
GFTISSFG

SEQ ID NO: 4 - protein, 8 aa, Artificial Sequence
  2A7 VH CDR2
ISSGSNTI

SEQ ID NO: 5 - protein, 6 aa, Artificial Sequence
  2A7 VH CDR3
ARRLAY

SEQ ID NO: 6 - protein, 5 aa, Artificial Sequence
  2A7 VL CDR1
SSVSS

SEQ ID NO: 7 - protein, 3 aa, Artificial Sequence
  2A7 VL CDR2
DTS

SEQ ID NO: 8 - protein, 9 aa, Artificial Sequence
  2A7 VL CDR3
LQWSSYPLT

SEQ ID NO: 9 - protein, 15 aa, Artificial Sequence
  Immunogen
RSRTPSLPTPPTREP

SEQ ID NO: 10 - protein, 441 aa, Artificial Sequence
  CTR4-tau
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 11 - protein, 441 aa, Artificial Sequence
  CTR4-T217A
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPAPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 12 - DNA, 390 bp, Artificial Sequence
  Nucleotide sequence encoding 2A7 VH
gaattcgaagttaagctgcagcagtctgggggaggcttagtgcagcctggagggtcccggaaactctcctgtgaagcctctggattcactatcagtagctttggaatgcactgggttcgtcaggctccagagaaggggctggaatgggtcgcatacattagtagtggcagtaataccatctactatgcagacacagtgaagggccgattcaccatctccagagacattcccaagaacaccctattcctgcaaatgaccagtctaaggtctgaggacacggccatgtattactgtgcaagacgactcgcttactggggccaagggactctggtcactgtctctgcagccaaaacaacacccccatcagtctatccactggcccctagatct

SEQ ID NO: 13 - DNA, 357 bp, Artificial Sequence
  Nucleotide sequence encoding 2A7 VL
gagctcgacattgtgctcacacaaactccagcaatcatgtctgcatctccaggggagaaggtcaccatgacctgcagtgccagctcaagtgtaagttccatgtactggtaccagcagaagccaggatcctcccccagactcctgatttttgacacatccaacctggcttctggagtccctgttcgcttcagtggcagtgggtctgggacctcttactctctcacaatcagccgaatggaggctgaagatgttgccacttattactgcctacagtggagtagttacccgctcacgttcggtgctgggaccaagctggagctgaaacgggctgatgctgcaccaactgtatccgcatgc

(19) United States
(12) Patent Application Publication
(10) Pub. No.: US 2025/0289874 A1
(43) Pub. Date: Sep. 18, 2025

(54) ANTIBODY AGAINST P-TAU 217 AND USE THEREOF

(71) Applicant: XIAMEN UNIVERSITY, Xiamen, Fujian (CN)

(72) Inventors: Yingjun ZHAO, Xiamen, Fujian (CN); Huaxi XU, Xiamen, Fujian (CN); Denghong ZHANG, Xiamen, Fujian (CN); Yunwu ZHANG, Xiamen, Fujian (CN)

(21) Appl. No.: 18/860,373

(22) PCT Filed: May 12, 2022
(86) PCT No.: PCT/CN2022/092435

§ 371 Date: Apr. 11, 2025

(30) Foreign Application Priority Data
Apr. 28, 2022 (CN) 202210460696.0

(51) Int. Cl. C07K 16/18; A61P 35/00

(52) U.S. Cl. CPC C07K 16/18; A61P 35/00; C07K 2317/31

(57) ABSTRACT

The present application belongs to the technical field of biomedicine, and more particularly, relates to an antibody or an antigen-binding fragment thereof capable of specifically binding to p-tau 217, and a multi-specific molecule, a pharmaceutical composition, and a kit comprising same. The present application further relates to use of the antibody or antigen-binding fragment thereof in preparing a kit or a drug. A monoclonal antibody (for example, 2A7 antibody) according to the present application has a high clinical application value in the detection and prevention of AD and the treatment of AD and other tau protein diseases.

### TECHNICAL FIELD

The present application belongs to the field of biomedical technology. More specifically, the present application relates to an antibody or antigen-binding fragment thereof capable of specifically binding to p-tau 217 (tau protein with phosphorylated amino acid at position 217), as well as a multispecific molecule, pharmaceutical composition and kit containing the same. The present application also relates to a use of the antibody or antigen-binding fragment thereof in the manufacture of a kit or medicament.

### BACKGROUND ART

Alzheimer's disease (AD) is the most common age-related neurodegenerative disease, and the prevalence gradually increases with age. According to the 2021 annual report of the Alzheimer's Association of the United States, there are currently about 6.2 million AD patients in the population over the age of 65 in the United States, and it is expected that the number would reach 13.8 million by 2060. With the aging of the population in China, AD and other forms of dementia are also increasing sharply. According to the 2020 meta-analysis by researchers at Shanxi Medical University, the current prevalence of AD in China is about 4%, and it is also found that the prevalence is closely related to gender, age, education level and region. AD not only seriously damages the health and quality of life of the elderly, but also brings huge economic burdens to the family and society.

The main pathological characteristics of AD include neuritic plaques formed by the deposition of β-amyloid protein (Aβ) and neurofibrillary tangles (NFTs) formed by the aggregation of hyperphosphorylated tau protein, activation of microglia and astrocytes, loss of synapses, and neuronal death. Its clinical symptoms include progressive memory loss, impaired executive function and difficulty in daily activities. The early stage of AD often manifests as changes in thinking or unconscious behavior, memory impairment for new information, and changes in language dysfunction. Late-stage AD patients will experience severe memory loss, hallucinations, disorientation and lack of self-care ability, which will eventually endanger their lives. Due to the complex etiology of AD and the lack of a clear pathological mechanism, there is currently no intervention method that can prevent or reverse the progression of AD, and all that can be achieved is temporary improvement or alleviation of the development of symptoms. Therefore, it is urgent to find new clinical therapeutic drugs.

The pathogenesis of AD is still unclear. Scientists have proposed a variety of hypotheses on the pathogenesis of AD, such as the Aβ cascade hypothesis, the cholinergic hypothesis, the tau protein abnormal phosphorylation hypothesis, the neuroinflammation hypothesis, the metal ion disorder hypothesis, etc. Based on these hypotheses, many drug researchers have developed drugs targeting different pathways and conducted corresponding clinical trials, but all have had little success. Currently, the anti-AD drugs on the market mainly include four categories, cholinesterase inhibitors (e.g., tacrine, donepezil, galantamine and rivastigmine, etc.), NMDA receptor antagonists (e.g., memantine), antibodies or inhibitors of Aβ and tau (e.g., Aducanumab, solanezumab and TPI-287, etc.), and intestinal flora regulators (e.g., GV-971). These drugs have a certain therapeutic effect on patients with early and mid-stage AD and can maintain the patient's cognitive state. However, these drugs cannot prevent the progression of AD patients, and they basically have serious side effects, such as hepatotoxicity of tacrine and gastrointestinal adverse reactions such as nausea and vomiting of rivastigmine. The research and development focuses of many pharmaceutical companies are mainly on the symptomatic clinical treatment stage, and there are relatively few studies on prevention. When AD patients show early clinical symptoms, the pathological changes in the body has already appeared. One of the main reasons for the poor efficacy of AD treatments is that the clinical treatment intervention time is too late. The current treatments are often started in the middle and late stages when the patients have obvious clinical symptoms, resulting in poor treatment effects. Therefore, early detection, diagnosis and effective risk prediction are crucial for the prevention and treatment of AD.

At present, the targets of AD treatment mainly include pathogenic Aβ and tau protein. However, a large number of drugs targeting Aβ failed in the clinical trial stage. For example, the clinical trials of γ-secretase inhibitors (semagacestat and avagacestat) did not show efficacy and were forced to stop due to increased incidence of skin cancer and infection. The trail of anti-Aβ vaccine AN1792 was terminated due to severe adverse reactions of aseptic meningitis. Therefore, researchers turned their attention to tau protein because it mediates neuronal dysfunction and death caused by Aβ. Tau is a microtubule-associated protein (encoded by MAPT gene) that participates in microtubule stability, and mainly enriched around neuronal axons. Its main functions include regulating and maintaining microtubule stability and assisting the transport function of neuronal axons. Excessive phosphorylation of tau protein will cause it to dissociate from microtubules and aggregate into neurotoxic oligomers and/or fibers, causing neuronal dysfunction and death. It is worth noting that in addition to AD, the deposition of phosphorylated tau protein is also the main pathological feature and pathogenic factor of a variety of neurodegenerative diseases, such as progressive superanuclear palsy (PSP), corticobasal degeneration (CBD) and frontotemporal dementia (FTD). These diseases are also called tauopathies. Therefore, therapeutic strategies targeting tau protein can be used for this large class of neurodegenerative diseases. At present, therapeutic drugs targeting tau protein mainly include tau protein targeted vaccines, small molecule tau-aggregation inhibitors, antisense oligonucleotides targeting genes encoding tau protein, and anti-tau protein antibodies. Tau protein is an intracellular protein, but it can be discharged into the spaces between brain cells as a free protein or in vesicles, where it diffuses and causes abnormal tau protein aggregation and irreversible damage. In an antibody-based immunotherapy, the diffused tau protein is bound by the antibody to delay or prevent neurodegeneration. However, most of the current anti-tau protein antibodies target the N-terminal antigen epitope, such as AC Immune's semorinemab (recognizes various forms of full-length tau protein), Biogen's gosuranemab and AbbVie's ABBV-8E12 (recognizes an epitope near the N-terminal). These antibodies are not able to specifically target pathogenic tau protein (e.g., phosphorylated tau protein), fail to achieve the maximum therapeutic effect, and most end in clinical failures. Therefore, the development of antibodies against pathogenic tau protein has important exploratory significance for the treatment of AD or other tauopathies.

Late intervention in AD is another important reason for the poor treatment effect of AD, so early diagnosis of AD is particularly important. Clinical diagnosis of AD is mainly based on clinical symptoms, neuropsychological tests, neuroimaging examinations, and cerebrospinal fluid tests. Among them, Aβ positron emission tomography (amyoid-PET-CT) is the gold standard for AD diagnosis, but it is not easy to screen the population due to price, equipment and venue restrictions. At the same time, neuropsychological tests are easily affected by factors such as the subject's life experience, education level, race and gender, and are usually used as auxiliary detection methods. The clinical symptoms of early AD patients are not significant and almost undetectable, and it is easy to miss the best time for diagnosis and treatment. Therefore, it is crucial to include AD biomarker detection in routine screening of the general population for the prevention and treatment of AD. Many countries in the world have included AD biomarker screening in the AD prevention and treatment guidelines. Among them, the changes in the content of AD-related biomarkers (Aβ40, Aβ42, t-tau and p-tau, etc.) in cerebrospinal fluid can directly reflect the damage of neurons and manifest about 20 years earlier than the onset of clinical symptoms. At present, tau protein phosphorylated at threonine 181 (p-tau 181) is the most widely used p-tau detection target, and it can be detected in serum samples. In 2020, Janelidze, Barthélemy and others found that in cerebrospinal fluid, p-tau 217 correlates better than p-tau 181 with tau-PET and neocortical Aβ plaque burden, and could more accurately distinguish AD from non-AD. Meanwhile, the sensitivity and specificity of p-tau 217 were greater than 90%. The accuracy of p-tau 217 in plasma in distinguishing clinical AD patients from other neurodegenerative disease patients (AUC=0.96) was significantly higher than that of p-tau 181, NfL and MRI detection (AUC=0.50 to 0.81). In addition, plasma p-tau 217 combined with APOE genotyping and cognitive examination can also be used in AD risk assessment models.

Studies have shown that aggregated tau protein is prone to N-terminal truncation due to protease action, so antibodies targeting a mid-segment of tau protein may have greater advantages. Since tau protein phosphorylated at position 217 can be detected in the early stage of AD, and a large amount of tau protein phosphorylated at position 217 has been detected in the insoluble components of brain tissue extracts of patients with AD or other tauopathies, targeting tau protein phosphorylated at position 217 may be able to intervene in the disease process in the early stage of AD or other tauopathies, which is of great significance.

However, so far, there have been no reports of using p-tau 217 as an early diagnostic criterion for AD and other tauopathies, nor of monoclonal antibody therapeutic drugs that specifically recognize tau protein phosphorylated at position 217. Therefore, establishing an efficient, highly sensitive, highly specific, and easy-to-operate early diagnostic method for AD or other tauopathies with pathological p-tau 217 as a target, and preparing more antibody therapeutic drugs that specifically recognize and block pathogenic phosphorylation of tau at position 217, will provide more possibilities for the treatment of tauopathies such as AD, and also bring new hope to patients.

### CONTENTS OF THE APPLICATION

Based on the deficiencies of the prior art, one of the main purposes of the present application is to provide an antibody capable of specifically binding to p-tau 217. The present application also provides a method for preparing the antibody and a use of the same. The anti-p-tau 217 antibody of the present application can be used for detection, prevention and/or treatment of a tauopathy, especially AD.

Therefore, in a first aspect, the present application provides an antibody or antigen-binding fragment thereof capable of specifically binding to p-tau 217, wherein the antibody or antigen-binding fragment thereof comprises:
(a) a heavy chain variable region (VH) comprising the following 3 complementarity determining regions (CDRs):
a VH CDR1, which consists of the following sequence: SEQ ID NO: 3, or a sequence having a substitution, deletion or addition of one or several amino acids (e.g., a substitution, deletion or addition of 1, 2 or 3 amino acids) as compared thereto,
a VH CDR2, which consists of the following sequence: SEQ ID NO: 4, or a sequence having a substitution, deletion or addition of one or several amino acids (e.g., a substitution, deletion or addition of 1, 2 or 3 amino acids) as compared thereto, and
a VH CDR3, which consists of the following sequence: SEQ ID NO: 5, or a sequence having a substitution, deletion or addition of one or several amino acids (e.g., a substitution, deletion or addition of 1, 2 or 3 amino acids) as compared thereto;
and/or,
(b) a light chain variable region (VL) comprising the following 3 complementarity determining regions (CDRs):
a VL CDR1, which consists of the following sequence: SEQ ID NO: 6, or a sequence having a substitution, deletion or addition of one or several amino acids (e.g., a substitution, deletion or addition of 1, 2 or 3 amino acids) as compared thereto,
a VL CDR2, which consists of the following sequence: SEQ ID NO: 7, or a sequence having a substitution, deletion or addition of one or several amino acids (e.g., a substitution, deletion or addition of 1, 2 or 3 amino acids) as compared thereto, and
a VL CDR3, which consists of the following sequence: SEQ ID NO: 8, or a sequence having a substitution, deletion or addition of one or several amino acids (e.g., a substitution, deletion or addition of 1, 2 or 3 amino acids) as compared thereto.

In certain embodiments, the substitution as described in any one of (i) to (vi) is a conservative substitution.

In certain embodiments, the CDRs described in any one of (i) to (vi) are defined according to the Kabat, Chothia or IMGT numbering system.

In certain embodiments, the CDRs described in any one of (i) to (vi) are defined according to the IMGT numbering system.

In certain embodiments, the antibody or antigen-binding fragment thereof comprises the following three CDRs of a heavy chain variable region: a VH CDR1 as set forth in SEQ ID NO: 3, a VH CDR2 as set forth in SEQ ID NO: 4, a VH CDR3 as set forth in SEQ ID NO: 5; and/or, the following three CDRs of a light chain variable region: a VL CDR1 as set forth in SEQ ID NO: 6, a VL CDR2 as set forth in SEQ ID NO: 7, a VL CDR3 as set forth in SEQ ID NO: 8.

In certain embodiments, the antibody or antigen-binding fragment thereof comprises:
(a) a heavy chain variable region (VH), which comprises an amino acid sequence selected from the group consisting of:
a sequence as set forth in SEQ ID NO: 1;
a sequence having a substitution, deletion or addition of one or several amino acids (e.g., a substitution, deletion or addition of 1, 2, 3, 4 or 5 amino acids) as compared to the sequence as set forth in SEQ ID NO: 1; or
a sequence having a sequence identity of at least 80%, at least 85%, at least 90%, at least 91%, at least 92%, at least 93%, at least 94%, at least 95%, at least 96%, at least 97%, at least 98%, at least 99%, or 100% as compared to the sequence as set forth in SEQ ID NO: 1;
and/or
(b) a light chain variable region (VL), which comprises an amino acid sequence selected from the group consisting of:
a sequence as set forth in SEQ ID NO: 2;
a sequence having a substitution, deletion or addition of one or several amino acids (e.g., a substitution, deletion or addition of 1, 2, 3, 4 or 5 amino acids) as compared to the sequence as set forth in SEQ ID NO: 2; or
a sequence having a sequence identity of at least 80%, at least 85%, at least 90%, at least 91%, at least 92%, at least 93%, at least 94%, at least 95%, at least 96%, at least 97%, at least 98%, at least 99%, or 100% as compared to the sequence as set forth in SEQ ID NO: 2.

In certain embodiments, the substitution described in (ii) or (v) is a conservative substitution.

In certain embodiments, the antibody or antigen-binding fragment thereof comprises a heavy chain framework region sequence and/or a light chain framework region sequence derived from a human immunoglobulin.

In certain embodiments, the antibody or antigen-binding fragment thereof comprises: a VH having a sequence as set forth in SEQ ID NO: 1 and a VL having a sequence as set forth in SEQ ID NO: 2.

In certain embodiments, the antibody or antigen-binding fragment thereof comprises a constant region or variant thereof derived from a human immunoglobulin.

In certain embodiments, the antibody or antigen-binding fragment thereof comprises:
a heavy chain constant region (CH) or variant thereof derived from a human immunoglobulin, wherein the variant has a substitution, deletion or addition of one or more amino acids or any combination thereof (e.g., a substitution, deletion or addition of up to 20, up to 15, up to 10, or up to 5 amino acids or any combination thereof; e.g., a substitution, deletion or addition of 1, 2, 3, 4 or 5 amino acids or any combination thereof) as compared to the sequence from which it is derived; and/or
a light chain constant region (CL) or variant thereof derived from a human immunoglobulin, wherein the variant has a substitution, deletion or addition of one or more amino acids or any combination thereof (e.g., a substitution, deletion or addition of up to 20, up to 15, up to 10, or up to 5 amino acids or any combination thereof; e.g., a substitution, deletion or addition of 1, 2, 3, 4 or 5 amino acids or any combination thereof) as compared to the sequence from which it is derived.

In certain embodiments, the heavy chain constant region is an IgG heavy chain constant region, such as an IgG1, IgG2, IgG3 or IgG4 heavy chain constant region.

In certain embodiments, the light chain constant region is a κ light chain constant region.

In certain embodiments, the antigen binding fragment is selected from the group consisting of Fab, Fab′, F(ab′)₂, Fv, disulfide-linked Fv, BsFv, dsFv, (dsFv)₂, dsFv-dsFv′, scFv, scFv dimer, camelized single chain domain antibody, diabody, ds diabody, nanobody, single domain antibody (sdAb), bivalent domain antibody; and/or, the antibody is a murine antibody, a chimeric antibody, a humanized antibody or a multispecific antibody.

The antibody of the present invention can be prepared by various methods known in the art, such as by genetic engineering recombinant technology. For example, DNA molecules encoding the heavy chain and light chain of the antibody of the present invention are obtained by chemical synthesis or PCR amplification. The obtained DNA molecules are inserted into an expression vector and then transfected into a host cell. Then, the transfected host cell is cultured under certain conditions and expresses the antibody of the present invention.

The antigen-binding fragment of the present invention can be obtained by hydrolyzing the intact antibody molecule (see, Morimoto et al., J. Biochem. Biophys. Methods 24:107-117 (1992) and Brennan et al., Science 229:81 (1985)). In addition, these antigen-binding fragments can also be directly produced by recombinant host cells (reviewed in Hudson, Curr. Opin. Immunol. 11:548-557 (1999); Little et al., Immunol. Today, 21: 364-370 (2000)). For example, Fab′ fragments can be directly obtained from host cells; Fab' fragments can be chemically coupled to form F(ab′)₂ fragments (Carter et al., Bio/Technology, 10: 163-167 (1992)). In addition, Fv, Fab or F(ab′)₂ fragments can also be directly isolated from the cell culture of recombinant host cells. Ordinary technicians in this field are fully aware of other techniques for preparing these antigen-binding fragments.

In certain embodiments, the antibody or antigen-binding fragment thereof is labeled. In certain embodiments, the antibody or antigen-binding fragment thereof is labeled with a detectable label, such as an enzyme (e.g., horseradish peroxidase), a radionuclide, a fluorescent dye, a luminescent substance (e.g., a chemiluminescent substance) or biotin.

In another aspect, the present application provides an isolated nucleic acid molecule encoding the antibody or antigen-binding fragment thereof as described above, or its heavy chain variable region and/or light chain variable region.

In certain embodiments, the nucleic acid molecule comprises a nucleotide sequence as set forth in SEQ ID NO: 12 or SEQ ID NO: 13.

In certain embodiments, the isolated nucleic acid molecule comprises a first nucleotide sequence encoding the heavy chain or heavy chain variable region of the antibody or antigen-binding fragment thereof of the present invention and a second nucleotide sequence encoding the light chain or light chain variable region of the antibody or antigen-binding fragment thereof, wherein the first nucleotide sequence and the second nucleotide sequence are present on the same or different isolated nucleic acid molecules. When the first nucleotide sequence and the second nucleotide sequence are present on different isolated nucleic acid molecules, the isolated nucleic acid molecule of the present invention comprises a first nucleic acid molecule comprising the first nucleotide sequence and a second nucleic acid molecule comprising the second nucleotide sequence.

In another aspect, the present application provides a vector, which comprises the nucleic acid molecule as described above. In certain embodiments, the vector is a cloning vector or an expression vector.

In certain embodiments, the vector comprises a first nucleotide sequence encoding the heavy chain or heavy chain variable region of the antibody or antigen-binding fragment thereof of the present invention and a second nucleotide sequence encoding the light chain or light chain variable region of the antibody or antigen-binding fragment thereof, wherein the first nucleotide sequence and the second nucleotide sequence are present on the same or different vectors. When the first nucleotide sequence and the second nucleotide sequence are present on different vectors, the vector of the present invention comprises a first vector comprising the first nucleotide sequence and a second vector comprising the second nucleotide sequence.

In another aspect, the present application provides a host cell, which comprises the nucleic acid molecule as described above or the vector as described above.

Such host cell includes, but is not limited to, prokaryotic cell such as bacterial cell (e.g., E. coli cell), and eukaryotic cell such as fungal cell (e.g., yeast cell), insect cell, plant cell and animal cell (e.g., mammalian cell, such as mouse cell, human cell, etc.).

In another aspect, the present application provides a method for preparing the antibody or antigen-binding fragment thereof as described above, comprising culturing the host cell as described above under a condition that allows expression of the antibody or antigen-binding fragment thereof, and recovering the antibody or antigen-binding fragment thereof from a culture of the cultured host cell. In certain embodiments, the host cell is a mammalian cell.

In another aspect, the present application provides a multispecific molecule, which comprises the antibody or antigen-binding fragment thereof as described above.

In certain embodiments, the multispecific molecule is capable of specifically binding to p-tau 217, and additionally specifically binding to one or more other targets.

In certain embodiments, the multispecific molecule further comprises at least one molecule (e.g., a second antibody or antigen-binding fragment thereof) having a binding specificity for a second target.

In certain embodiments, the multispecific molecule comprises the antibody or antigen-binding fragment thereof as described above, and a second antibody or antigen-binding fragment thereof.

In certain embodiments, the multispecific molecule comprises the antibody or antigen-binding fragment thereof as described above, and a second antibody or antigen-binding fragment thereof linked thereto.

In another aspect, the present application provides a pharmaceutical composition, which comprises the antibody or antigen-binding fragment thereof as described above, or the multispecific molecule as described above, and a pharmaceutically acceptable carrier and/or excipient.

In certain embodiments, the pharmaceutical composition further comprises an additional pharmaceutically active agent.

In certain embodiments, the additional pharmaceutically active agent is a drug having activity for treating a tauopathy (e.g., AD).

In certain exemplary embodiments, the pharmaceutically acceptable carrier and/or excipient comprises a sterile injectable liquid (e.g., an aqueous or non-aqueous suspension or solution). In certain exemplary embodiments, such sterile injectable liquid is selected from the group consisting of water for injection (WFI), bacteriostatic water for injection (BWFI), sodium chloride solution (e.g., 0.9% (w/v) NaCl), glucose solution (e.g., 5% glucose), surfactant-containing solution (e.g., a solution containing 0.01% polysorbate 20), pH buffer solution (e.g., phosphate buffer solution), Ringer's solution, and any combination thereof.

In another aspect, the present application provides a kit, which comprises the antibody or antigen-binding fragment thereof as described above.

In some embodiments, the antibody or antigen-binding fragment thereof carries a detectable label, such as an enzyme (e.g., horseradish peroxidase), a radionuclide, a fluorescent dye, a luminescent substance (e.g., a chemiluminescent substance), or biotin.

In some embodiments, the kit further comprises a second antibody capable of specifically recognizing the antibody or antigen-binding fragment thereof as described above.

In some embodiments, the second antibody further comprises a detectable label, such as an enzyme (e.g., horseradish peroxidase), a radionuclide, a fluorescent dye, a luminescent substance (e.g., a chemiluminescent substance), or biotin.

In some embodiments, the kit is used to detect the presence or amount of p-tau 217 in a sample.

In some embodiments, the sample is a cerebrospinal fluid, whole blood, serum, or plasma obtained from a subject.

In some embodiments, the kit further comprises a reagent (e.g., horse serum) for diluting the sample.

In some embodiments, the second antibody is coated on magnetic beads.

In some embodiments, the subject is a mammal, such as a human.

In another aspect, the present application provides a method for preventing and/or treating a tauopathy in a subject (e.g., a human), the method comprising administering to the subject in need thereof an effective amount of the antibody or antigen-binding fragment thereof as described above, or the multispecific molecule as described above, or the pharmaceutical composition as described above.

In certain embodiments, the tauopathy includes, but is not limited to, Alzheimer's disease (AD), primary age-related tauopathy, chronic traumatic encephalopathy, Pick's disease, and corticobasal degeneration.

In certain embodiments, the tauopathy is selected from the group consisting of Alzheimer's disease (AD), primary age-related tauopathy, chronic traumatic encephalopathy, Pick's disease, and corticobasal degeneration.

In certain embodiments, the tauopathy is AD.

In certain embodiments, the cerebrospinal fluid of the subject comprises p-tau 217.

In certain embodiments, the subject is a mammal, such as a human.

In certain embodiments, the method further comprises administering an additional drug having the activity of preventing and/or treating AD.

In another aspect, the present application provides a use of the antibody or antigen-binding fragment thereof as described above, or the multispecific molecule as described above, or the pharmaceutical composition as described above, in the manufacture of a medicament for preventing and/or treating a tauopathy in a subject (e.g., a human).

In certain embodiments, the tauopathy includes, but is not limited to, Alzheimer's disease (AD), primary age-related tauopathy, chronic traumatic encephalopathy, Pick's disease, and corticobasal degeneration.

In certain embodiments, the tauopathy is selected from the group consisting of Alzheimer's disease (AD), primary age-related tauopathy, chronic traumatic encephalopathy, Pick's disease, and corticobasal degeneration.

In certain embodiments, the tauopathy is AD.

In certain embodiments, the medicament further comprises an additional pharmaceutically active agent that is active in treating a tauopathy (e.g., AD). In certain embodiments, the cerebrospinal fluid of the subject comprises p-tau 217.

In certain embodiments, the subject is a mammal, such as a human.

In another aspect, the present application provides the antibody or antigen-binding fragment thereof as described above, or the multispecific molecule as described above, or the pharmaceutical composition as described above, for preventing and/or treating a tauopathy in a subject (e.g., a human).

In certain embodiments, the tauopathy includes, but is not limited to, Alzheimer's disease (AD), primary age-related tauopathy, chronic traumatic encephalopathy, Pick's disease, and corticobasal degeneration.

In certain embodiments, the tauopathy is selected from the group consisting of Alzheimer's disease (AD), primary age-related tauopathy, chronic traumatic encephalopathy, Pick's disease, and corticobasal degeneration.

In certain embodiments, the tauopathy is AD.

In certain embodiments, the antibody or antigen-binding fragment thereof as described above, or the multispecific molecule as described above, or the pharmaceutical composition as described above is administered in combination with an additional pharmaceutically active agent that is active in treating tauopathy (e.g., AD).

In certain embodiments, the cerebrospinal fluid of the subject comprises p-tau 217.

In certain embodiments, the subject is a mammal, such as a human.

In another aspect, the present application provides a method for detecting the presence or amount of p-tau 217 in a sample, comprising the following steps:
contacting the sample with the antibody or antigen-binding fragment thereof as described above;
detecting the formation of a complex of the antibody or antigen-binding fragment thereof and p-tau 217 or detecting the amount of the complex.

In certain embodiments, the antibody or antigen-binding fragment thereof carries a detectable label.

In certain embodiments, the method is performed in a subject or in vitro.

In another aspect, the present application provides a use of the antibody or antigen-binding fragment thereof as described above, or the multispecific molecule as described above, in the manufacture of a reagent for detecting whether a subject suffers from a tauopathy, or for distinguishing a patient with Alzheimer's disease (AD) from a patient with other tauopathy.

In certain embodiments, the tauopathy includes, but is not limited to, Alzheimer's disease (AD), primary age-related tauopathy, chronic traumatic encephalopathy, Pick's disease, and corticobasal degeneration.

In some embodiments, the tauopathy is selected from the group consisting of Alzheimer's disease (AD), primary age-related tauopathy, chronic traumatic encephalopathy, Pick's disease, and corticobasal degeneration.

In some embodiments, the tauopathy is AD.

In some embodiments, the reagent detects the amount of p-tau 217 in the sample by the method as described above to detect whether the subject suffers from tauopathy, or to distinguish the patient with Alzheimer's disease (AD) from the patient with other tauopathy, and the sample is obtained from the subject or patient.

In some embodiments, the sample is a blood sample (e.g., whole blood, serum, plasma).

In another aspect, the present application provides a method for detecting whether a subject suffers from a tauopathy, or distinguishing a patient with Alzheimer's disease (AD) from a patient with other tauopathy, the method comprising:
detecting the amount of p-tau 217 in a sample by using the antibody or antigen-binding fragment thereof as described above, and the sample is obtained from the subject or patient;
optionally, the method further comprises comparing the detected amounts of p-tau 217 in different samples to detect whether the subject suffers from Alzheimer's disease (AD), or to distinguish the patient with Alzheimer's disease (AD) from the patient with other tauopathy.

In certain embodiments, the method comprises:
(1) contacting the sample with the antibody or antigen-binding fragment thereof as described above;
(2) detecting the formation of a complex of the antibody or antigen-binding fragment thereof and p-tau 217 or detecting the amount of the complex.

In certain embodiments, the sample is a blood sample (e.g., whole blood, serum, plasma).

In certain embodiments, the tauopathy includes, but is not limited to, Alzheimer's disease (AD), primary age-related tauopathy, chronic traumatic encephalopathy, Pick's disease, and corticobasal degeneration.

In some embodiments, the tauopathy is selected from the group consisting of Alzheimer's disease (AD), primary age-related tauopathy, chronic traumatic encephalopathy, Pick's disease, and corticobasal degeneration.

In some embodiments, the tauopathy is AD.

In another aspect, the present application provides the antibody or antigen-binding fragment thereof as described above, or the multispecific molecule as described above, for detecting whether a subject has a tauopathy, or for distinguishing a patient with Alzheimer's disease (AD) from a patient with other tauopathy.

In some embodiments, the tauopathy includes, but is not limited to, Alzheimer's disease (AD), primary age-related tauopathy, chronic traumatic encephalopathy, Pick's disease, and corticobasal degeneration.

In some embodiments, the tauopathy is selected from the group consisting of Alzheimer's disease (AD), primary age-related tauopathy, chronic traumatic encephalopathy, Pick's disease, and corticobasal degeneration.

In some embodiments, the tauopathy is AD.

In certain embodiments, the reagent detects the amount of p-tau 217 in a sample to detect whether the subject suffers from Alzheimer's disease (AD), or to distinguish the patient with Alzheimer's disease (AD) from the patient with other tauopathy, and the sample is obtained from the subject or patient.

### Definition of Terms

In the present application, unless otherwise specified, the scientific and technical terms used herein have the meanings commonly understood by those skilled in the art. In addition, the molecular genetics, nucleic acid chemistry, chemistry, molecular biology, biochemistry, cell culture, microbiology, cell biology, genomics and recombinant DNA operation steps used herein are all routine steps widely used in the corresponding fields. At the same time, in order to better understand the present application, the definitions and explanations of the relevant terms are provided below.

As used herein, the term “p-tau 217” refers to a phosphorylated tau protein, which is phosphorylated at the amino acid at position 217 corresponding to the native tau protein. Since pathological p-tau 217 can be detected in the early stage of AD, and a large amount of p-tau 217 can be detected in the insoluble components of brain tissue extracts of AD patients, antibodies targeting p-tau 217 have great application potential in the prevention, detection and treatment of AD. In certain embodiments, the natural tau protein has an amino acid sequence as set forth in SEQ ID NO: 10.

As used herein, the term “natural tau protein” refers to a biologically active, naturally occurring tau protein. The amino acid sequence of the natural tau protein can be easily obtained from various public databases (e.g., GenBank database). In certain embodiments, the natural tau protein has an amino acid sequence as set forth in SEQ ID NO: 10.

As used herein, when referring to the amino acid sequence of the natural tau protein, it is described using the sequence as set forth in SEQ ID NO: 10. For example, the expression “the amino acid at position 127 of the natural tau protein” refers to the amino acid at position 127 of the protein as set forth in SEQ ID NO: 10. However, those skilled in the art understand that the natural tau protein may have multiple versions, which have substantially the same primary structure (i.e., amino acid sequence) and higher-order structure (i.e., spatial structure), and substantially the same biological function, but they may still have slight differences in amino acid sequence. Therefore, in the present application, the natural tau protein is not limited to the protein as set forth in SEQ ID NO: 10, but is intended to cover all known natural tau proteins. Therefore, in the present application, the term “natural tau protein” should include various naturally occurring tau proteins with biological function, including, for example, the tau protein as set forth in SEQ ID NO: 10 and its naturally occurring variants. Moreover, when describing the amino acid position of tau protein, it includes not only the specific amino acid position in SEQ ID NO: 10, but also the amino acid position corresponding to the specific amino acid position in its natural variant. For example, the expression “amino acid at position 127 of natural tau protein” includes the amino acid at position 127 of SEQ ID NO: 10, and the amino acid at the corresponding position in its natural variant. According to the present application, the expression “corresponding amino acid position” refers to the amino acid position in the compared sequences, which is at the equivalent position when the sequences are optimally aligned, i.e., when the sequences are aligned to obtain the highest percentage identity. Similarly, the expression “corresponding to position 127 of SEQ ID NO: 10” refers to the amino acid position in the compared sequence, which is at the position equivalent to position 127 of SEQ ID NO: 10 when the sequence is optimally aligned with SEQ ID NO: 10, i.e., when the sequence is aligned with SEQ ID NO: 10 to obtain the highest percentage identity.

As used herein, the term “identity” is used to refer to the matching of sequences between two polypeptides or between two nucleic acids. When a position in both compared sequences is occupied by the same nucleotide or amino acid residue (e.g., a position in each of two DNA molecules is occupied by adenine nucleotide, or a position in each of two polypeptides is occupied by lysine), then the molecules are identical at that position. “Percentage identity” between two sequences is a function of the number of matching positions shared by the two sequences divided by the number of positions compared×100. For example, if 6 out of 10 positions of two sequences match, then the two sequences have an identity of 60%. For example, the DNA sequences CTGACT and CAGGTT have an identity of 50% (3 out of a total of 6 positions match). Typically, the comparison is made when the two sequences are aligned to produce maximum identity. Such an alignment can be achieved by using, for example, the method of Needleman et al. (1970) J. Mol. Biol. 48: 443-453, which can be conveniently performed by a computer program such as the Align program (DNAstar, Inc.). The percentage identity between two amino acid sequences can also be determined using the algorithm of E. Meyers and W. Miller (Comput. Appl Biosci., 4:11-17 (1988)), which is incorporated into the ALIGN program (version 2.0), using a PAM120 weight residue table, a gap length penalty of 12, and a gap penalty of 4. In addition, the percentage identity between two amino acid sequences can be determined using the algorithm of Needleman and Wunsch (J Mol Biol. 48:444-453 (1970)), which is incorporated into the GAP program of the GCG software package (available at www.gcg.com), using a Blossum 62 matrix or a PAM250 matrix, a gap weight of 16, 14, 12, 10, 8, 6, or 4 and a length weight of 1, 2, 3, 4, 5, or 6.

As used herein, the term “tauopathies” refers to diseases caused by abnormalities (e.g., abnormal aggregation) of the microtubule-associated protein tau. In certain embodiments, tauopathy is a disease caused by abnormal aggregation or deposition of pathological tau protein in neurons or glial cells. Alzheimer's disease is the most representative tauopathy.

As used herein, the term “phosphorylation” refers to the addition of a phosphate group to an amino acid residue of a protein. Typically, amino acid residues such as threonine, serine, and tyrosine have hydroxyl groups and are therefore easily phosphorylated.

As used herein, the term “antibody” refers to an immunoglobulin molecule typically composed of two pairs of polypeptide chains (each pair having a light chain (LC) and a heavy chain (HC)). Antibody light chains can be classified as κ (kappa) and λ (lambda) light chains. Heavy chains can be classified as μ, δ, γ, α, or ε, and the isotype of antibody is defined as IgM, IgD, IgG, IgA, and IgE, respectively. In the light chain and heavy chain, variable region and constant region are connected by a “J” region of about 12 or more amino acids, and the heavy chain also contains a “D” region of about 3 or more amino acids. Each heavy chain consists of a heavy chain variable region (VH) and a heavy chain constant region (CH). The heavy chain constant region consists of 3 domains (CH1, CH2 and CH3). Each light chain consists of a light chain variable region (VL) and a light chain constant region (CL). The light chain constant region consists of one domain CL. The constant domain does not directly participate in the binding of antibody to antigen, but exhibits a variety of effector functions, such as mediating the binding of immunoglobulin to host tissue or factor, including various cells of immune system (e.g., effector cells) and first component of classical complement system (C1q). The VH and VL regions can also be subdivided into regions with high variability (called complementarity determining regions (CDRs)), interspersed with more conservative regions called framework regions (FRs). Each VH and VL consists of three CDRs and four FRs arranged from the amino terminus to the carboxyl terminus in the following order: FR1, CDR1, FR2, CDR2, FR3, CDR3, FR4. The variable regions (VH and VL) of each heavy chain/light chain pair form antigen binding sites, respectively. The allocation of amino acids in each region or domain can follow the definitions of Kabat, Sequences of Proteins of Immunological Interest (National Institutes of Health, Bethesda, Md. (1987 and 1991)), or Chothia & Lesk (1987) J. Mol. Biol. 196:901-917; Chothia et al. (1989) Nature 342:878-883.

As used herein, the term “complementarity determining region” or “CDR” refers to the amino acid residues in a variable region of an antibody that are responsible for antigen binding. There are three CDRs in each of the variable regions of the heavy and light chains, designated as CDR1, CDR2, and CDR3. The precise boundaries of these CDRs can be defined according to various numbering systems known in the art, such as the Kabat numbering system (Kabat et al., Sequences of Proteins of Immunological Interest, 5th Ed. Public Health Service, National Institutes of Health, Bethesda, Md., 1991), the Chothia numbering system (Chothia & Lesk (1987) J. Mol. Biol. 196:901-917; Chothia et al. (1989) Nature 342:878-883) or the IMGT numbering system (Lefranc et al., Dev. Comparat. Immunol. 27:55-77, 2003). For a given antibody, a person skilled in the art will readily identify the CDRs defined by each numbering system. Moreover, the correspondence between different numbering systems is well known to those skilled in the art (for example, see Lefranc et al., Dev. Comparat. Immunol. 27:55-77, 2003).

In the present application, the CDRs contained in the antibody or antigen-binding fragment thereof of the present invention can be determined according to various numbering systems known in the art. In certain embodiments, the CDRs contained in the antibody or antigen-binding fragment thereof of the present invention are preferably determined by the Kabat, Chothia or IMGT numbering systems.

As used herein, the term “framework region” or “FR” residues refers to those amino acid residues in a variable region of an antibody other than the CDR residues defined above.

As used herein, the terms “monoclonal antibody”, “McAb”, and “mAb” have the same meaning and are used interchangeably, and refer to an antibody or a fragment of an antibody from a group of highly homologous antibody molecules, that is, a group of identical antibody molecules except for natural mutations that may occur spontaneously. Monoclonal antibodies have high specificity for a single epitope on an antigen. Polyclonal antibodies are relative to monoclonal antibodies, which usually contain at least 2 or more different antibodies, which usually recognize different epitopes on an antigen. In addition, the modifier “monoclonal” only indicates that the antibody is characterized by being obtained from a highly homologous antibody group, and it cannot be understood that the antibody needs to be prepared by any specific method.

The monoclonal antibodies of the present invention can be prepared by a variety of techniques, such as hybridoma technology (see, for example, Kohler et al., Nature, 256:495, 1975), recombinant DNA technology (see, for example, U.S. Pat. No. 4,816,567), or phage antibody library technology (see, for example, Clackson et al. Nature 352:624-628, 1991, or Marks et al. J. Mol. Biol. 222:581-597, 1991).

As used herein, the term “antigen-binding fragment” of an antibody refers to a polypeptide comprising a fragment of a full-length antibody that retains the ability to specifically bind to the same antigen that the full-length antibody binds to and/or competes with the full-length antibody for specific binding to the antigen. It is also referred to as “antigen-binding portion”. See generally, Fundamental Immunology, Ch.7 (Paul, W., ed., 2nd edition, Raven Press, N.Y. (1989), which is incorporated herein by reference in its entirety for all purposes. Antigen binding fragments of antibodies can be produced by recombinant DNA technology or by enzymatic or chemical cleavage of intact antibodies. Non-limiting examples of antigen binding fragments include Fab, Fab′, F(ab′)₂, Fd, Fv, complementarity determining region (CDR) fragment, scFv, diabody, single domain antibody, chimeric antibody, linear antibody, nanobody (technology from Domantis), probody and such polypeptides, which contain at least a portion of an antibody that is sufficient to confer the polypeptide a specific antigen binding ability. Engineered antibody variants are reviewed in Holliger et al., 2005; Nat Biotechnol, 23: 1126-1136.

As used herein, the term “full-length antibody” refers to an antibody consisting of two “full-length heavy chains” and two “full-length light chains”. Wherein, “full-length heavy chain” refers to a polypeptide chain that is composed of a heavy chain variable region (VH), a heavy chain constant region CH1 domain, a hinge region (HR), a heavy chain constant region CH2domain, and a heavy chain constant region CH3 domain in the direction from N-terminal to C-terminal; and, when the full-length antibody is an IgE isotype, it optionally also comprises a heavy chain constant region CH4 domain. Preferably, a “full-length heavy chain” is a polypeptide chain consisting of VH, CH1, HR, CH2 and CH3 in the direction from N-terminal to C-terminal. A “full-length light chain” is a polypeptide chain consisting of a light chain variable region (VL) and a light chain constant region (CL) in the N-terminal to C-terminal direction. Two pairs of full-length antibody chains are linked together by disulfide bonds between CL and CH1 and between the HRs of two full-length heavy chains. The full-length antibody of the present invention may be from a single species, such as a human; and it may also be a chimeric antibody or a humanized antibody. The full-length antibody of the present invention comprises two antigen binding sites formed by two VH and VL pairs, respectively, which specifically recognize/bind to the same antigen.

As used herein, the term “Fd” refers to an antibody fragment consisting of VH and CH1domains; the term “dAb fragment” refers to an antibody fragment consisting of a VH domain (Ward et al., Nature 341:544 546 (1989)); the term “Fab fragment” refers to an antibody fragment consisting of VL, VH, CL and CH1 domains; the term “F(ab′)₂ fragment” refers to an antibody fragment comprising two Fab fragments linked by a disulfide bridge at hinge region; the term “Fab′ fragment” refers to a fragment that is obtained after reduction of the disulfide bonds linking the two heavy chain fragments in F(ab′)₂ fragment, and consists of a complete light chain and an Fd fragment of heavy chain (consisting of VH and CH1 domains).

As used herein, the term “Fv” refers to an antibody fragment consisting of the VL and VH domains of a single arm of an antibody. The Fv fragment is generally considered to be the smallest antibody fragment that can form a complete antigen binding site. It is generally believed that the six CDRs confer antigen binding specificity to an antibody. However, even a single variable region (e.g., Fd fragment, which contains only three CDRs specific for an antigen) can recognize and bind to the antigen, although its affinity may be lower than that of the complete binding site.

As used herein, the term “Fc” refers to an antibody fragment formed by disulfide bonding of the second and third constant regions of the first heavy chain of an antibody to the second and third constant regions of the second heavy chain. The Fc fragment of an antibody has a variety of different functions but does not participate in antigen binding.

As used herein, the term “scFv” refers to a single polypeptide chain comprising VL and VH domains, wherein the VL and VH are linked by a linker (see, for example, Bird et al., Science 242:423-426 (1988); Huston et al., Proc. Natl. Acad. Sci. USA 85:5879-5883 (1988); and Pluckthun, The Pharmacology of Monoclonal Antibodies, Vol. 113, Roseburg and Moore, eds., Springer-Verlag, New York, pp. 269-315 (1994)). Such scFv molecules may have the general structure: NH₂-VL-linker-VH-COOH or NH₂-VH-linker-VL-COOH. Suitable prior art linkers consist of repeated GGGGS amino acid sequences or variants thereof. For example, a linker having the amino acid sequence (GGGGS)₄ may be used, but variants thereof may also be used (Holliger et al. (1993), Proc. Natl. Acad. Sci. USA 90: 6444-6448). Other linkers that can be used in the present invention are described by Alfthan et al. (1995), Protein Eng. 8:725-731, Choi et al. (2001), Eur. J. Immunol. 31: 94-106, Hu et al. (1996), Cancer Res. 56:3055-3061, Kipriyanov et al. (1999), J. Mol. Biol. 293:41-56 and Roovers et al. (2001), Cancer Immunol. In some cases, a disulfide bond may also exist between the VH and VL of scFv. In certain embodiments of the present invention, the scFv may form a di-scFv, which refers to that two or more single scFvs are linked in series to form an antibody. In certain embodiments of the present invention, the scFv may form a (scFv)₂, which refers to that two or more single scFvs are linked in parallel to form an antibody.

As used herein, the term “single-domain antibody (sdAb)” has the meaning generally understood by those skilled in the art, which refers to an antibody fragment composed of a single monomeric variable antibody domain (e.g., a single heavy chain variable region) that retains the ability to specifically bind to the same antigen bound by the full-length antibody. The single-domain antibody is also called nanobody.

Each of the above antibody fragments retains the ability to specifically bind to the same antigen bound by the full-length antibody, and/or competes with the full-length antibody for specific binding to the antigen.

Antigen-binding fragments of antibodies (e.g., the above antibody fragments) can be obtained from a given antibody (e.g., the antibody provided by the present invention) using conventional techniques known to those skilled in the art (e.g., recombinant DNA technology or enzymatic or chemical cleavage methods), and the antigen-binding fragments of antibodies can be screened for specificity in the same manner as for intact antibodies.

As used herein, unless the context clearly indicates otherwise, when referring to the term “antibody”, it includes not only intact antibody, but also antigen-binding fragments of the antibody.

As used herein, the term “chimeric antibody” refers to an antibody in which a portion of the light chain or/and heavy chain is derived from one antibody (which may be derived from a particular species or belong to a particular antibody class or subclass), and another portion of the light chain or/and heavy chain is derived from another antibody (which may be derived from the same or different species or belong to the same or different antibody class or subclass), but in any case, it still retains binding activity to the target antigen (U.S. Pat. No. 4,816,567 to Cabilly et al.; Morrison et al., Proc. Natl. Acad. Sci. USA, 81:6851 6855 (1984)). In certain embodiments, the term “chimeric antibody” may include an antibody in which the heavy chain and light chain variable regions of the antibody are derived from a first antibody, and the heavy chain and light chain constant regions of the antibody are derived from a second antibody.

As used herein, the term “variant”, in the context of polypeptide (including a polypeptide), also refers to a polypeptide or peptide comprising an amino acid sequence that has been altered by introducing a substitution, deletion or addition of amino acid residues. In some cases, the term “variant” also refers to a polypeptide or peptide that has been modified (i.e., by covalently attaching any type of molecule to the polypeptide or peptide). For example, but not limiting, the polypeptide can be modified, for example, by glycosylation, acetylation, pegylation, phosphorylation, amidation, derivatization with known protecting/blocking groups, proteolytic cleavage, attachment to cellular ligands or other proteins, etc. Derivatized polypeptides or peptides can be produced by chemical modification using techniques known to those skilled in the art, including but not limited to specific chemical cleavage, acetylation, formylation, metabolic synthesis of tunicamycin, etc. In addition, the variant has similar, identical or improved functions to the polypeptide or peptide from which it is derived.

As used herein, the term “specific binding” refers to a non-random binding reaction between two molecules, such as a reaction between an antibody and an antigen to which it is directed. The strength or affinity of a specific binding interaction can be expressed by the equilibrium dissociation constant (K_{D}) of the interaction. In the present application, the term “K_{D}” refers to the dissociation equilibrium constant of a specific antibody-antigen interaction, which is used to describe the binding affinity between an antibody and an antigen. The smaller the equilibrium dissociation constant, the tighter the antibody-antigen binding and the higher the affinity between the antibody and the antigen.

The specific binding properties between two molecules can be determined using methods known in the art. One method involves measuring the rate of formation and dissociation of an antigen binding site/antigen complex. Both the “association rate constant” (kₐ or kₒₙ) and the “dissociation rate constant” (k_{dis} or k_{off}) can be calculated from concentrations and the actual rates of association and dissociation (see, Malmqvist M, Nature, 1993, 361:186-187). The ratio of k_{dis}/kₒₙ is equal to the dissociation constant K_{D} (see, Davies et al., Annual Rev Biochem, 1990; 59:439-473). K_{D}, kₒₙ and k_{dis} values can be measured by any effective method. In some embodiments, the dissociation constant can be measured by surface plasmon resonance (SPR) with a Biacore instrument. In addition, the dissociation constant can be measured by bioluminescence interferometry or Kinexa.

As used herein, the detectable label of the present application can be any substance detectable by fluorescent, spectroscopic, photochemical, biochemical, immunological, electrical, optical or chemical means. Such labels are well known in the art, and examples thereof include, but are not limited to, enzymes (e.g., horseradish peroxidase, alkaline phosphatase, β-galactosidase, urease, glucose oxidase, etc.), radionuclides (e.g., ³H, ¹²⁵I, ³⁵S, ¹⁴C or ³²P), fluorescent dyes (e.g., fluorescein isothiocyanate (FITC), fluorescein, tetramethylrhodamine isothiocyanate (TRITC), phycoerythrin (PE), Texas Red, rhodamine, quantum dots or cyanine dye derivatives (e.g., Cy7, Alexa 750)), luminescent substances (e.g., chemiluminescent substances, such as acridinium ester compounds, luminol and derivatives thereof, ruthenium derivatives such as terpyridine ruthenium), magnetic beads (e.g., Dynabeads®), calorimetric labels such as colloidal gold or colored glass or plastic (e.g., polystyrene, polypropylene, latex, etc.) beads, and biotin for binding to avidin (e.g., streptavidin) modified with the above labels.

As used herein, the term “vector” refers to a nucleic acid vehicle into which a polynucleotide can be inserted. When a vector can express the protein encoded by the inserted polynucleotide, the vector is called an expression vector. The vector can be introduced into a host cell by transformation, transduction or transfection so that the genetic material elements it carries are expressed in the host cell. Vectors are well known to those skilled in the art, and include but are not limited to: plasmid; phagemid; cosmid; artificial chromosome, such as yeast artificial chromosome (YAC), bacterial artificial chromosome (BAC) or P1-derived artificial chromosome (PAC); bacteriophage such as λ phage or M13 phage and animal virus. Animal viruses that can be used as vectors include but are not limited to retrovirus (including lentivirus), adenovirus, adeno-associated virus, herpes virus (e.g., herpes simplex virus), poxvirus, baculovirus, papillomavirus, papovavirus (e.g., SV40). A vector can contain a variety of elements that control expression, including but not limited to promoter sequence, transcription start sequence, enhancer sequence, selection element and reporter gene. In addition, the vector may also contain a replication origin.

As used herein, the term “host cell” refers to a cell that can be used to introduce a vector, including but not limited to prokaryotic cell such as Escherichia coli or Bacillus subtilis, fungal cell such as yeast cell or Aspergillus, insect cell such as S2 Drosophila cell or Sf9, or animal cell such as fibroblast, CHO cell, COS cell, NSO cell, HeLa cell, BHK cell, HEK 293 cell or human cell.

As used herein, the term “conservative substitution” refers to an amino acid substitution that does not adversely affect or change the expected properties of the protein/polypeptide comprising the amino acid sequence. For example, conservative substitutions can be introduced by standard techniques known in the art, such as site-directed mutagenesis and PCR-mediated mutagenesis. Conservative amino acid substitutions include substitutions of amino acid residues with amino acid residues having similar side chains, such as substitutions with residues that are physically or functionally similar to the corresponding amino acid residues (e.g., having similar size, shape, charge, chemical properties, including the ability to form covalent bonds or hydrogen bonds, etc.). Families of amino acid residues with similar side chains have been defined in the art. These families include amino acids with basic side chains (e.g., lysine, arginine, and histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), β-branched side chains (e.g., threonine, valine, isoleucine), and aromatic side chains (e.g., tyrosine, phenylalanine, tryptophan, histidine). Thus, it is preferred to replace a corresponding amino acid residue with another amino acid residue from the same side chain family. Methods for identifying conservative amino acid substitutions are well known in the art (see, for example, Brummell et al., Biochem. 32:1180-1187 (1994)).93); Kobayashi et al. Protein Eng. 12(10):879-884 (1999); and Burks et al. Proc. Natl Acad. Set USA 94:412-417 (1997), which are incorporated herein by reference).

The twenty conventional amino acids referred to herein are written in accordance with conventional usage. See, for example, Immunology-A Synthesis (2nd Edition, E. S. Golub and D. R. Gren, Eds., Sinauer Associates, Sunderland, Mass. (1991)), which is incorporated herein by reference. In the present application, the terms “polypeptide” and “protein” have the same meaning and are used interchangeably. And in the present application, amino acids are generally represented by single-letter and three-letter abbreviations known in the art. For example, alanine can be represented by A or Ala.

As used herein, the term “pharmaceutically acceptable carrier and/or excipient” refers to a carrier and/or excipient that is pharmacologically and/or physiologically compatible with the subject and the active ingredient, which is well known in the art (see, for example, Remington's Pharmaceutical Sciences. Edited by Gennaro AR, 19th ed. Pennsylvania: Mack Publishing Company, 1995), and includes, but is not limited to, pH regulator, surfactant, adjuvant, ionic strength enhancer, diluent, agent for maintaining osmotic pressure, agent for delaying absorption, preservative. For example, the pH regulator includes, but is not limited to, phosphate buffer. The surfactant includes, but is not limited to, cationic, anionic or nonionic surfactant, such as Tween-80. The ionic strength enhancer includes, but is not limited to, sodium chloride. The preservative includes, but is not limited to, various antibacterial and antifungal agents, such as paraben, chlorobutanol, phenol, sorbic acid, and the like. The agent for maintaining osmotic pressure includes, but is not limited to, sugar, NaCl, and the like. The agent for delaying absorption includes, but is not limited to, monostearate and gelatin. The diluent includes, but is not limited to, water, aqueous buffer (e.g., buffered saline), alcohol and polyol (e.g., glycerol), etc. The preservative includes, but is not limited to, various antibacterial and antifungal agent, such as thimerosal, 2-phenoxyethanol, paraben, chlorobutanol, phenol, sorbic acid, etc. The stabilizer has the meanings generally understood by those skilled in the art, which can stabilize the desired activity of the active ingredient in the drug, including but not limited to sodium glutamate, gelatin, SPGA, sugar (e.g., sorbitol, mannitol, starch, sucrose, lactose, dextran, or glucose), amino acid (e.g., glutamic acid, glycine), protein (e.g., dried whey, albumin or casein) or degradation product thereof (e.g., lactalbumin hydrolysate), etc. In certain exemplary embodiments, the pharmaceutically acceptable carrier or excipient includes a sterile injectable liquid (e.g., an aqueous or non-aqueous suspension or solution). In certain exemplary embodiments, such sterile injectable liquid is selected from the group consisting of water for injection (WFI), bacteriostatic water for injection (BWFI), sodium chloride solution (e.g., 0.9% (w/v) NaCl), glucose solution (e.g., 5% glucose), surfactant-containing solution (e.g., a solution containing 0.01% polysorbate 20), pH buffer solution (e.g., phosphate buffer solutions), Ringer's solution, and any combination thereof.

As used herein, the term “prevention” refers to a method performed to prevent or delay the occurrence of a disease or disorder or symptom in a subject. As used herein, the term “treatment” refers to a method performed to obtain a beneficial or desired clinical outcome. For the purposes of the present application, the beneficial or desired clinical outcome includes (but is not limited to) alleviation of symptom, reduction of the scope of disease, stabilization (i.e., no longer worsening) of the state of disease, delay or slowing of the progression of disease, improvement or alleviation of the state of disease, and relief of symptom (whether partial or complete), whether detectable or undetectable. In addition, “treatment” may also refer to prolonging survival compared to the expected survival if not receiving treatment.

As used herein, the term “subject” refers to a mammal, such as a human, a cynomolgus monkey, or a mouse. In certain embodiments, the subject (e.g., human, cynomolgus monkey, or mouse) suffers from a disease associated with TIGIT (e.g., a tumor involving TIGIT-positive infiltrating T cells and/or NK cells, and/or a tumor involving TIGIT ligand (e.g., CD155 and/or CD112)-positive tumor cells), or is at risk of suffering from the above-mentioned disease.

As used herein, the term “effective amount” refers to an amount sufficient to obtain or at least partially obtain the desired effect. For example, an effective amount for preventing a disease (e.g., a tumor involving TIGIT-positive infiltrating T cells and/or NK cells, and/or a tumor involving TIGIT ligand (e.g., CD155 and/or CD112)-positive tumor cells) refers to an amount sufficient to prevent, arrest, or delay the occurrence of the disease; an effective amount for treating a disease refers to an amount sufficient to cure or at least partially prevent the disease and its complications in a patient already suffering from the disease. Determining such an effective amount is well within the capabilities of those skilled in the art. For example, the effective amount for therapeutic use will depend on the severity of the disease to be treated, the overall state of the patient's own immune system, the patient's general condition such as age, weight and gender, the mode of administration of the drug, and other treatments administered concurrently, etc.

### Beneficial Effects of the Present Invention

The monoclonal antibody (e.g., 2A7 antibody) of the present application is capable of binding to p-tau 217 with high specificity. At the same time, the monoclonal antibody of the present application can also be used for detecting the content of p-tau 217 (e.g., the content of p-tau 217 in the cerebrospinal fluid of a subject), so the monoclonal antibody can be used to identify or detect AD patients and distinguish AD patients from other tauopathy patients.

Administering the monoclonal antibody to a subject with tauopathy can improve the behavior and ability of the subject (e.g., significantly increase the time exploring new objects, improve the perception of surrounding light and spatial avoidance ability, improve spatial learning and memory), inhibit the atrophy of hippocampus of the subject, and improve the pathological changes of brain tissue. Therefore, the monoclonal antibody (e.g., 2A7 antibody) of the present application has a high clinical application value in the detection and prevention of AD, and the treatment of AD and other tauopathies.

The embodiments of the present invention will be described in detail below with reference to the drawings and examples, but those skilled in the art will understand that the following drawings and examples are only used to illustrate the present invention, rather than to limit the scope of the present invention. According to the following detailed description of the drawings and preferred embodiments, the various objects and advantages of the present invention will become apparent to those skilled in the art.

### BRIEF DESCRIPTION OF THE DRAWINGS

FIG. 1 shows the immunoblotting results of antibody 2A7 on different proteins in part 2.1 of Example 2, with GAPDH as an internal reference.

FIG. 2 shows the tissue immunofluorescence staining results of antibody 2A7 on different proteins in part 2.1 of Example 2; therein, the binding areas of 2A7 or the irrelevant monoclonal antibody were shown in green, NeuN-specific antibody binding areas were shown in red, and DAPI binding areas were shown in blue.

FIG. 3 shows the immunoblotting results of antibody 2A7 on different proteins in part 2.2 of Example 2, with GAPDH as an internal reference.

FIG. 4 shows the results of detecting p-tau 217 contents in cerebrospinal fluid of AD patients and PS19 mice by 2A7 antibody in Example 3.

FIG. 5 shows the behavioral improvement results of 10.5-month-old PS19 mice after treatment with 2A7 antibody. Among them, panel A shows that in the new object recognition test, the time spent by the mice exploring new objects in the PS19-2A7 group was significantly increased as compared with that of the PS19-IgG group; panel B shows that in the open field test, after treatment with 2A7 antibody, the time spent by PS19 mice in the middle of the box was significantly reduced, indicating that the perception of surrounding light and spatial avoidance ability in mice were improved; panels C and D show that in the Morris water maze experiment, the spatial learning and memory ability of the mice in the PS19-2A7 group was significantly improved compared with that of the PS19-IgG group.

FIG. 6 shows the results of the inhibition of hippocampal atrophy in 10.5-month-old PS19 mice after treatment with 2A7 antibody. Among them, panel A shows the results of MRI detection of axial anatomical structure (T1) and 3D reconstruction of the brain of mice in the WT-IgG, PS19-IgG and PS19-2A7 groups; panel B shows the statistical results of hippocampal volume of mice in the WT-IgG, PS19-IgG and PS19-2A7 groups.

FIG. 7 shows the results of pathological alleviation in 10.5-month-old PS19 mice after treatment with 2A7 antibody. Among them, the immunofluorescence staining results in panel A shows that the loss of neurons (NeuN) in mice in the PS19-2A7 group was significantly reduced; panel B shows the statistical results of NeuN fluorescence intensity in mice in the WT-IgG, PS19-IgG and PS19-2A7 groups; panel C shows that the staining signal of p-tau 217 (2A7) in mice in the PS19-2A7 group was significantly reduced; panel D shows the statistical results of 2A7-based staining fluorescence intensity in mice in the WT-IgG, PS19-IgG and PS19-2A7 groups; panel E shows that the proliferation of microglia (IBA1) in mice in the PS19-2A7 group was significantly inhibited; panel F shows the statistical results of IBA1 fluorescence intensity in mice in the WT-IgG, PS19-IgG and PS19-2A7 groups.

Note: Quantification results are shown as mean±SEM. Statistical analysis was performed using GraphPad Prism software (version 9.0, https://www.graphpad.com/). Where appropriate, differences were evaluated by unpaired t-test or one-way ANOVA. P value<0.05 was considered statistically significant. * represents P<0.05, ** represents P<0.01, and *** represents P<0.001.

### SEQUENCE INFORMATION

The information of the partial sequences involved in the present application is provided in Table 1 below.

TABLE 1
Description of sequences

| SEQ | | |
|---|---|---|
| ID NO: | Description | Sequence |
| 1 | 2A7 VH | EFEVKLQQSGGGLVQPGGSRKLSCEASGFTISSFGMHWVR |
| | | QAPEKGLEWVAYISSGSNTIYYADTVKGRFTISRDIPKNTLF |
| | | LQMTSLRSEDTAMYYCARRLAYWGQGTLVTVSAAKTTPP |
| | | SVYPLAPRS |
| 2 | 2A7 VL | ELDIVLTQTPAIMSASPGEKVTMTCSASSSVSSMYWYQQK |
| | | PGSSPRLLIFDTSNLASGVPVRFSGSGSGTSYSLTISRMEAE |
| | | DVATYYCLQWSSYPLTFGAGTKLELKRADAAPTVSAC |
| 3 | 2A7 VH | GFTISSFG |
| | CDR1 | |
| 4 | 2A7 VH | ISSGSNTI |
| | CDR2 | |
| 5 | 2A7 VH | ARRLAY |
| | CDR3 | |
| 6 | 2A7 VL CDR1 | SSVSS |
| 7 | 2A7 VL CDR2 | DTS |
| 8 | 2A7 VL CDR3 | LQWSSYPLT |
| 9 | Immunogen | RSRTPSLPTPPTREP |
| 10 | CTR4-tau | MAEPRQEFEVMEDHAGTYGLGDRKDQGGYTMHQDQEGD |
| | | TDAGLKESPLQTPTEDGSEEPGSETSDAKSTPTAEDVTAPL |
| | | VDEGAPGKQAAAQPHTEIPEGTTAEEAGIGDTPSLEDEAAG |
| | | HVTQARMVSKSKDGTGSDDKKAKGADGKTKIATPRGAAP |
| | | PGQKGQANATRIPAKTPPAPKTPPSSGEPPKSGDRSGYSSPG |
| | | SPGTPGSRSRTPSLPTPPTREPKKVAVVRTPPKSPSSAKSRL |
| | | QTAPVPMPDLKNVKSKIGSTENLKHQPGGGKVQIINKKLD |
| | | LSNVQSKCGSKDNIKHVPGGGSVQIVYKPVDLSKVTSKCG |
| | | SLGNIHHKPGGGQVEVKSEKLDFKDRVQSKIGSLDNITHVP |
| | | GGGNKKIETHKLTFRENAKAKTDHGAEIVYKSPVVSGDTS |
| | | PRHLSNVSSTGSIDMVDSPQLATLADEVSASLAKQGL |
| 11 | CTR4-T217A | MAEPRQEFEVMEDHAGTYGLGDRKDQGGYTMHQDQEGD |
| | | TDAGLKESPLQTPTEDGSEEPGSETSDAKSTPTAEDVTAPL |
| | | VDEGAPGKQAAAQPHTEIPEGTTAEEAGIGDTPSLEDEAAG |
| | | HVTQARMVSKSKDGTGSDDKKAKGADGKTKIATPRGAAP |
| | | PGQKGQANATRIPAKTPPAPKTPPSSGEPPKSGDRSGYSSPG |
| | | SPGTPGSRSRTPSLPAPPTREPKKVAVVRTPPKSPSSAKSRL |
| | | QTAPVPMPDLKNVKSKIGSTENLKHQPGGGKVQIINKKLD |
| | | LSNVQSKCGSKDNIKHVPGGGSVQIVYKPVDLSKVTSKCG |
| | | SLGNIHHKPGGGQVEVKSEKLDFKDRVQSKIGSLDNITHVP |
| | | GGGNKKIETHKLTFRENAKAKTDHGAEIVYKSPVVSGDTS |
| | | PRHLSNVSSTGSIDMVDSPQLATLADEVSASLAKQGL |
| 12 | Nucleotide | GAATTCGAAGTTAAGCTGCAGCAGTCTGGGGGAGGCTT |
| | sequence | AGTGCAGCCTGGAGGGTCCCGGAAACTCTCCTGTGAAG |
| | encoding 2A7 | CCTCTGGATTCACTATCAGTAGCTTTGGAATGCACTGGG |
| | VH | TTCGTCAGGCTCCAGAGAAGGGGCTGGAATGGGTCGCA |
| | | TACATTAGTAGTGGCAGTAATACCATCTACTATGCAGAC |
| | | ACAGTGAAGGGCCGATTCACCATCTCCAGAGACATTCCC |
| | | AAGAACACCCTATTCCTGCAAATGACCAGTCTAAGGTCT |
| | | GAGGACACGGCCATGTATTACTGTGCAAGACGACTCGCT |
| | | TACTGGGGCCAAGGGACTCTGGTCACTGTCTCTGCAGCC |
| | | AAAACAACACCCCCATCAGTCTATCCACTGGCCCCTAGA |
| | | TCT |
| 13 | Nucleotide | GAGCTCGACATTGTGCTCACACAAACTCCAGCAATCATG |
| | sequence | TCTGCATCTCCAGGGGAGAAGGTCACCATGACCTGCAGT |
| | encoding 2A7 | GCCAGCTCAAGTGTAAGTTCCATGTACTGGTACCAGCAG |
| | VL | AAGCCAGGATCCTCCCCCAGACTCCTGATTTTTGACACA |
| | | TCCAACCTGGCTTCTGGAGTCCCTGTTCGCTTCAGTGGC |
| | | AGTGGGTCTGGGACCTCTTACTCTCTCACAATCAGCCGA |
| | | ATGGAGGCTGAAGATGTTGCCACTTATTACTGCCTACAG |
| | | TGGAGTAGTTACCCGCTCACGTTCGGTGCTGGGACCAAG |
| | | CTGGAGCTGAAACGGGCTGATGCTGCACCAACTGTATCC |
| | | GCATGC |

### SPECIFIC MODELS FOR CARRYING OUT THE INVENTION

The present invention will now be described with reference to the following examples which are intended to illustrate the present invention (but not to limit the present invention).

Unless otherwise specified, the experiments and methods described in the examples were performed substantially according to conventional methods well known in the art and described in various references. For example, conventional techniques of immunology, biochemistry, chemistry, molecular biology, microbiology, cell biology, genomics and recombinant DNA used in the present application can be found in Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel et al., eds., (1987)); METHODS IN ENZYMOLOGY series (Academic Publishing Company): PCR 2: A PRACTICAL METHOD. APPROACH) (M. J. MacPherson, B. D. Hames and G. R. Taylor, ed. (1995)), and ANIMAL CELL CULTURE (R. I. Freshney, ed. (1987)).

In addition, if the specific conditions were not specified in the examples, they were carried out according to conventional conditions or conditions recommended by the manufacturer. The reagents or instruments used without indicating the manufacturer were all conventional products that could be obtained commercially. It is known to those skilled in the art that the examples describe the present invention by way of example and are not intended to limit the scope sought to be protected by the present invention. All the disclosures and other references mentioned herein are incorporated herein by reference in their entirety.

### Specific Models for Carrying Out the Invention

The present invention will now be described with reference to the following examples which are intended to illustrate the present invention (but not to limit the present invention).

Unless otherwise specified, the experiments and methods described in the examples were performed substantially according to conventional methods well known in the art and described in various references. For example, conventional techniques of immunology, biochemistry, chemistry, molecular biology, microbiology, cell biology, genomics and recombinant DNA used in the present application can be found in Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel et al., eds., (1987)); METHODS IN ENZYMOLOGY series (Academic Publishing Company): PCR 2: A PRACTICAL METHOD. APPROACH) (M. J. MacPherson, B. D. Hames and G. R. Taylor, ed. (1995)), and ANIMAL CELL CULTURE (R. I. Freshney, ed. (1987)).

In addition, if the specific conditions were not specified in the examples, they were carried out according to conventional conditions or conditions recommended by the manufacturer. The reagents or instruments used without indicating the manufacturer were all conventional products that could be obtained commercially. It is known to those skilled in the art that the examples describe the present invention by way of example and are not intended to limit the scope sought to be protected by the present invention. All the disclosures and other references mentioned herein are incorporated herein by reference in their entirety.

### EXAMPLE 1. PREPARATION OF MONOCLONAL ANTIBODIES CAPABLE OF SPECIFICALLY RECOGNIZING PHOSPHORYLATED SITE 217

In this study, a polypeptide phosphorylated at amino acid at position 217 was used for immunization, and a polypeptide non-phosphorylated at position 217 was used for differential screening to screen specific antibodies.

### (a) Preparation of Immunogen

The immunogen was KLH-C-RSRTPSLPT (p) PPTREP, which was a phosphorylated polypeptide with an amino acid sequence, as shown in SEQ ID NO: 9, corresponding to the amino acid sequence of positions 209-223 of the natural tau protein. The polypeptides used in this study were obtained by chemical synthesis, and synthesized by Nanjing Zoonbio Biotechnology Co., Ltd.

### (a) Experimental Mice

6-week-old SPF female Balb/C mice.

### (a) Preparation of Hybridomas

The hybridoma cells secreting monoclonal antibodies were obtained using standard in vivo immunization and PEG fusion methods. For detailed methods, see Ed Harlow et al., “Antibodies A Laboratory Manual”, Cold Spring Harbor Laboratory 1988. The brief process was described as follows:
1. Mouse immunization: First, 100 μg of polypeptide was mixed and emulsified with an equal volume of Complete Freund's adjuvant (CFA), and then the mice were first immunized by multiple injections into the muscles of limbs. Secondly, 50 μg of polypeptide was mixed and emulsified with an equal volume of Incomplete Freund's adjuvant (IFA), and then the mice were subjected to booster immunization on the 14^{th}, 28^{th} and 42^{nd} days after the first immunization. Finally, the mice were subjected to booster immunization by intraperitoneal immunization on the 56^{th} day after the first immunization, and the immunogen was an equal volume mixture of 50 μg polypeptide and PBS. Three days after the end of immunization, the spleens of the mice were taken for fusion experiment.
2. Cell fusion: The spleen of the mouse was taken, and ground to obtain a spleen cell suspension, which was then mixed with mouse myeloma cells SP2/0 in the logarithmic growth phase, and cell fusion was performed under the action of PEG1500. The fused cells were resuspended in 300 mL of fusion medium (RPMI-1640 medium containing HAT and 20% FBS) and divided into fifteen 96-well cell culture plates for culture.
3. Screening of hybridomas: The fused cells were cultured on the 96-well cell culture plates for 7 to 10 days, and then the cell supernatants were pipetted for ELISA detection. The polypeptide used for detection was a polypeptide phosphorylated at site 217. For the ELISA-positive cell wells, ELISA-based differential detection was performed with a polypeptide non-phosphorylated at site 217. The positive clones that reacted to the polypeptide phosphorylated at site 217 and did not react to the polypeptide non-phosphorylated at site 217 were cloned three times (differential screening was performed for each cloning test) to obtain a hybridoma cell line that could stably secrete antibodies. Finally, the 2A7 cell line that expresses the monoclonal antibody against the polypeptide phosphorylated at site 217 was obtained.
4. Hybridoma culture: The stable monoclonal hybridoma cell line was expanded and cultured in a carbon dioxide incubator, and transferred from the 96-well plate to a 24-well plate, a 6-well plate, and a 10 cm cell plate in turn. Then the cells in the cell plate were collected and injected into the mouse peritoneal cavity. After 7 to 10 days, the ascites containing the monoclonal antibody was extracted from the mouse peritoneal cavity.

(b) Monoclonal antibody purification: The mouse ascites containing the monoclonal antibody was treated with a 50% saturated ammonium sulfate solution. The obtained precipitate was then dissolved in PBS and purified using a Protein A column to obtain a purified monoclonal antibody, and the purity of the obtained monoclonal antibody was determined by SDS-PAGE.

The polynucleotide encoding the obtained monoclonal antibody was amplified by PCR, and the PCR product was sent to the company for sequencing to obtain the sequence. The obtained antibody was named 2A7, and its specific sequence was shown in Table 1, wherein the CDR sequence of the antibody was determined by the IMGT numbering system (Lefranc et al., Dev. Comparat. Immunol. 27:55-77, 2003).

### EXAMPLE 2. IDENTIFICATION OF SPECIFICITY OF 2A7

### 2.1 Reactivity and Specificity of 2A7 to Natural Tau Protein

The reactivity and specificity of 2A7 to natural tau protein were verified by Western Blot (WB) on the brain tissue of PS19 mice. Brain tissues of 6-month-old wild-type (WT) mice (containing murine tau protein), tau gene KO wild-type mice (Tau-KO) (not containing tau protein), and PS19 mice (transgenic mice overexpressing human tau with P301S mutation and expressing excessive phosphorylated human tau) were obtained. Among which, the brain tissues of WT and Tau-KO mice were resuspended in TNEN lysis buffer (20 mM Tris-HCl pH=7.4, 100 mM NaCl, 1 mM EDTA, 0.5% NP40), and lysed after adding a protease inhibitor and a phosphatase inhibitor. After lysis, centrifugation was performed at 12000 rpm for 10 min at 4° C., and the supernatant was stored at −80° C. for later use. PS19 mouse brain tissue was also resuspended in TNEN lysis buffer, lysed after adding a protease inhibitor, and centrifuged, and the supernatant was divided into three equal parts, in which the first part was added with a phosphatase inhibitor and stored at −80° C. for later use; the second part was added with a phosphatase inhibitor, mixed evenly, and a part was taken out and incubated at 37° C. for 1 h; and the third part was added with an alkaline phosphatase, mixed evenly, and a part was taken out and incubated at 37° C. for 1 h. 15 μg or more of the mouse brain lysate was taken to perform WB and verify the reactivity and specificity of 2A7 to native tau, with GAPDH as the internal reference. 2A7 was used at a concentration of 1 μg/mL, and HRP-labeled horse anti-mouse IgG (HAM-HRP, CST, 7076S) diluted 1:1000 was used for development (Clinx Science Instruments Co., Ltd, ChemiScope 6200).

The results were shown in FIG. 1. In FIG. 1, 2A7 had a strong reactivity to PS19 mouse brain lysate (as shown in lane 3 (PS19) and lane 5 (PS19, 37° C.) in FIG. 1). After alkaline phosphatase treatment, the reactivity was significantly reduced (as shown in lane 4 (PS19+CIP) in FIG. 1). At the same time, it was only weakly reactive to WT mice and WT mice with Tau gene KO (as shown in lane 1 (WT) and lane 2 (Tau-KO) in FIG. 1, respectively).

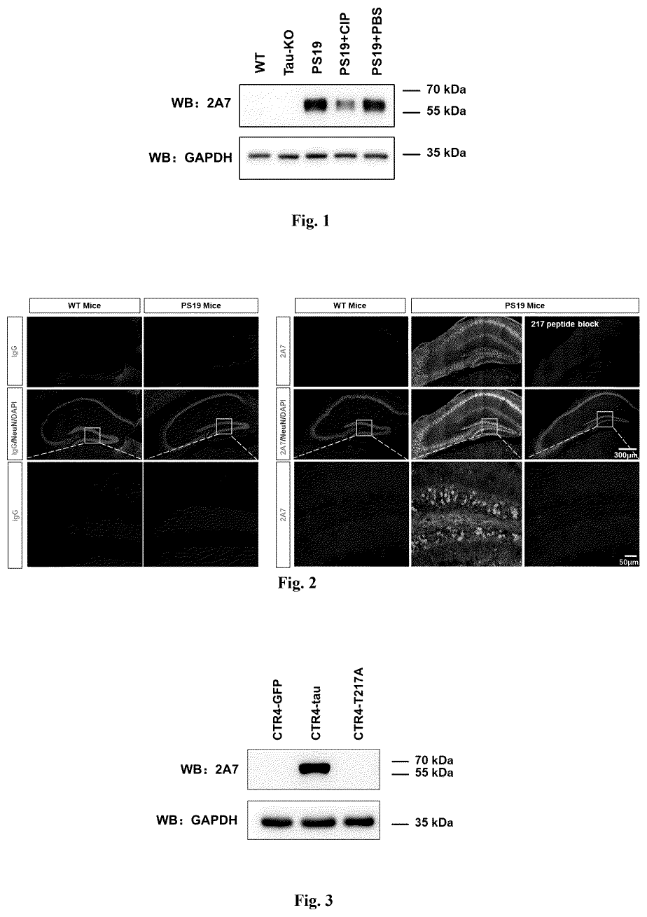

The reactivity and specificity of 2A7 were verified by tissue immunofluorescence staining. In the experiment, 6 groups were set, AD-irrelevant IgG (corresponding to the second column of IgG in FIG. 2), 2A7 antibody (corresponding to the fourth column of p-tau 217 in FIG. 2), and 2A7 antibody blocked with the polypeptide phosphorylated at 217 site (corresponding to the fifth column of 217 peptide block in FIG. 2) were separately used to stain the brain tissue sections of PS19 mice; and AD-irrelevant IgG antibody (corresponding to the first column of IgG in FIG. 2) and 2A7 antibody (corresponding to the third column of p-tau 217 in FIG. 2) were separately used to stain the brain tissue sections of WT mice. Among them, the above antibodies were used in an amount of 1 μg and the polypeptides were used in an amount of 5 μg. The brain tissues of WT and PS19 mice aged 13 months were taken for frozen sections. Two slices of WT mouse brain tissue and four slices of PS19 mouse brain tissue were washed three times with PBST, each washing for 10 minutes, respectively. Then, they were blocked with PBST containing 10% donkey serum (Solarbo, SL050) and 0.3% Triton X-100, respectively, and incubated at room temperature for 1 hour. The above antibodies were separately added to 200 uL of blocking solution, added to the corresponding mouse brain tissue, and incubated at 4° C. overnight. The above antibodies were recovered, and the brain tissue slices were washed 3 times with PBST, each washing for 10 minutes. The fluorescent secondary antibody and DAPI were diluted in the blocking solution at a ratio of 1:500 and 1:1000, respectively, and added to the corresponding mouse brain tissue slices, and incubated at room temperature in the dark for 1 hour. After that, the fluorescently labeled secondary antibody was removed, and the brain slices were washed 3 times with PBST in the dark, each washing for 10 minutes. The slices were sealed with anti-fluorescence quencher and stored in the dark at 4° C. Finally, Zeiss 880 was used to take pictures.

As shown in FIG. 2, compared with WT mice and irrelevant antibodies, the 2A7 antibody could specifically recognize the tau protein in PS19 hippocampus and cortex. After incubation with the polypeptide phosphorylated at site 217, the binding ability of the 2A7 antibody to the tau protein in the PS19 hippocampus and cortex was reduced.

### 2.2 Reactivity and Specificity of 2A7 to 217 Site Mutant of Natural Tau Protein

CTR4-Tau and CTR4-T217A vector with 217 phosphorylation site mutation were constructed respectively. The insert fragment of CTR4-Tau vector was a nucleotide sequence encoding tau (as set forth in SEQ ID NO: 10), and the insert fragment of CTR4-T217A vector was a nucleotide sequence encoding tau mutated at position 217 (as set forth in SEQ ID NO: 11). 293T cells were transfected with the constructed CTR4-Tau and CTR4-T217A vectors, respectively, and the cells were collected after 48 hours. PLCDH-GFP was used as a control. TNEN (containing protease inhibitor and phosphatase inhibitor) was used for lysis. After lysis, the cells were centrifuged at 4° C., 12000 rpm for 10 min, and the supernatant was taken to measure the concentration and stored at −80° C. for later use.

After the concentration of the above supernatant was determined, 15 μg was taken for WB to verify the specificity of 2A7, with β-actin as the internal reference. The concentration of 2A7 was 1 μg/mL, and GAM-HRP diluted 1:1000 was used as the secondary antibody for development.

The amino acid at position 217 of tau expressed by eukaryotic cells would be phosphorylated by a kinase in the cells, so it could be recognized by 2A7 antibody; while the mutation at position 217 was to mutate the amino acid T at position 217 to A, and the amino acid at this position could not be phosphorylated after the mutation. The results were shown in FIG. 3. The reactivity of 2A7 to the tau expressed by eukaryotic cells (lane 2 in FIG. 3) was higher than that to the tau after the mutation at position 217 (lane 3 in FIG. 3).

### EXAMPLE 3. DETECTION OF P-TAU 217 IN CEREBROSPINAL FLUIDS OF AD PATIENTS AND PS19 MICE BY 2A7 ANTIBODY

In this example, tauopathy model mice, i.e., PS19 mice, were used for experiment. Cerebrospinal fluid (CSF) of 13-month-old PS19 mice was obtained. For the collection of cerebrospinal fluid, reference was made to the optimized method for collecting cerebrospinal fluid from mice, as described by Lim et al. (for details, please refer to, Lim, N. K., V. Moestrup, X. Zhang, W. A. Wang, A. Moller and F. D. Huang (2018). “An Improved Method for Collection of Cerebrospinal Fluid from Anesthetized Mice.” J Vis Exp(133)), in which the mice were anesthetized, the dura mater on cistern magnum of the mice was exposed by surgery, the dura mater was punctured using the tip of a glass capillary under a stereoscope, avoiding blood vessels, to pipette cerebrospinal fluid, and then the cerebrospinal fluid was collected in a 1.5 mL centrifuge tube with protease inhibitor and phosphatase inhibitor, and stored at −80° C. for later use. The cerebrospinal fluids from 16 AD patients were obtained from Suzhou Astrabio Co., Ltd.

The detection kit (Cat. No.: Lite-P64050, Suzhou Astrabio) was used to detect the contents of p-tau 217 in the cerebrospinal fluids of AD and PS19 mice. The mouse cerebrospinal fluids were diluted 5 times with horse serum, and the AD cerebrospinal fluid samples were tested at their original concentrations. The reaction and analysis of the samples were all performed on Ast-Dx90 single molecule immunodiagnostic instrument.

The reagent calibration and quality control processes were completed according to the operating steps of Sc-lite single molecule immunoassay instrument. The samples and reagents were loaded to the designated positions in sequence. When ready, the test was started. The equipment automatically sent the samples to the loading positions, and the reaction cup was loaded into the incubation tray. 25 μL of sample was pipetted from the 96-well plate using a sampling needle and added to the reaction cup. 25 μL of capture antibody-coated magnetic bead solution (Reagent 1) was pipetted from the kit using a reagent needle and added to the reaction cup. They were mixed and incubated for 6 minutes.

10 μL of detection antibody modified with single molecule signal label (Reagent 2) was pipetted from the kit using the reagent needle and added to the reaction cup, mixed and incubated for 4 minutes, in which Reagent 2 comprised the single molecule signal label that modified the detection antibody, which could convert the target molecule into single molecule signal.

The reaction system was transferred to a flow cell using a detection needle, the magnetic beads were attracted to the bottom of the flow cell by magnetic separation and spread on the surface of detection well, after other components were removed by washing, an integrated fluorescence microscope was used to take fluorescent images, single molecule signals were analyzed by machine, and the biomarker concentration was calculated using a pre-prepared standard curve.

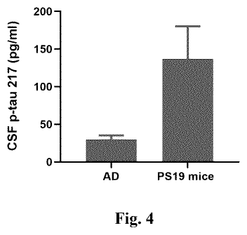

The experimental results were shown in FIG. 4, which showed that the use of 2A7 antibody could detect the content of p-tau 217 in samples such as cerebrospinal fluids of AD patients and cerebrospinal fluids of PS19 mice.

### EXAMPLE 4. NASAL ADMINISTRATION OF 2A7 ANTIBODY IN PS19 MICE

In this study, nasal administration was adopted to detect the improvement effect of 2A7 antibody on the pathology and behavior of PS19 mice (tauopathy model mice).

### 4.1 Antibody Preparation

The prepared 2A7 antibody and AD-irrelevant IgG were diluted with PBS to concentrations of 0.5 μg/μL and 1 μg/μL, respectively, aliquoted and stored at −80° C.

### 4.2 Experimental Mice and Grouping

In the experiment, 5-month-old SPF male PS19 mice, 20˜25 g, were divided into 3 groups: WT mice nasally administered with IgG (WT-IgG), PS19 mice nasally administered with IgG (PS19-IgG), and PS19 mice nasally administered with 2A7 antibody (PS19-2A7), 13 mice in each group.

### 4.3 Nasal Administration and Dosage of Antibodies

The mice in the experiment were all nasally administered. After being anesthetized with isoflurane, they were placed supinely, and the antibody was slowly dripped into the nostrils with a microsyringe. The mouth was closed during the dripping to facilitate the absorption of solution.

The antibody was administered once every 3 days for 5 consecutive months. The volume of drug administrated to each mouse was 20 μl, and each mouse was administered with 10 μg for the first three months and 20 μg for the next two months.

### EXAMPLE 5. BEHAVIORAL CHANGES IN PS19 MICE AFTER TREATMENT WITH 2A7 ANTIBODY

The behavioral experiments of all mice treated in Example 4 were performed using Smart Video Tracking Software (Panlab, Harvard Apparatus) for data acquisition and analysis. The animal behavior experiment was conducted between 9:00 a.m. and 7:00 p.m. every day, and the light intensity in the laboratory was 650 lux.
a. The tester touched the mice three days before the start of the experiment, once a day. Every time, the tester touched one mouse by gently grabbing the mouse and allowing the mouse stay in the tester's hand for 30 seconds, and marked the mouse's tail with a marker pen. After the mouse was marked, the tester grabbed its tail and gently put it back into the mouse cage;
b. On the day of the experiment, the mice to be tested were transferred to the experimental room before the experiment to allow the mice to adapt to the surrounding environment and light; before the experiment was ready to start, the box and maze used in the experiment were cleaned with 75% ethanol. After each round of experiment, 75% ethanol was used to wipe the box and maze to remove the feces and urine excreted by the mice during the experiment, and to eliminate the interference of the residual smell of the mice on the test results.

### 5.1 Open Field

The open field experiment was used to study the autonomous movement ability and anxiety behavior of mice, mainly based on the avoidance of mice to bright light and open space. The internal area of the open field box (40 cm long×40 cm wide×40 cm high) was divided into 16 small grids, the surrounding 12 grids were defined as the peripheral region, and the middle 4 grids were defined as the central region (Center); the mice were placed in the center of the open field box, and each mouse was placed in the same position (same grid, same direction); the mice were allowed to explore freely in the maze for 5 minutes, and the total movement distance of the mice in the maze and the time they were active in the central region of the maze (Time in center) were recorded.

### 5.2 New Object Recognition

The new object recognition experiment was a learning and memory test method established based on the principle that rodents were naturally curious about new objects.

On the first day, mice were placed in the center of an open field box (40 cm long×40 cm wide×40 cm high), in which each mouse was placed in the same position (same grid, same direction), and the mice were allowed to adapt for 5 min; on the second day, two identical objects A and B were placed on one side of the open field box, the mice were gently placed in the open field box to face the box wall so that the distances between the position to place the mice and the two objects were the same as far as possible, and the mice were allowed to explore freely for 8 min; on the third day, the object positions were kept unchanged, and one of the old objects (A or B) was replaced with a new object C; the mice were placed in the open field box to the box wall so that the distances between the position to place the mice and the two objects were the same as far as possible, and the mice were allowed to explore freely for 8 min. The mouse nose touching the objects or the nose pointing to the objects within 2 cm of the objects was considered as exploratory behavior; the camera system was used to record the time the mouse explored the familiar object and the new object.

### 5.3 Water Maze Test

Morris water maze experiment was used to study and evaluate the spatial learning and memory ability of mice.

The water maze test was conducted in a circular water tank (60 cm radius×100 cm height). The height of water in the water tank should be 2 cm above the platform, and the water temperature in the water tank was set to 22° C. Four icons of different shapes were posted in the four directions (E, S, W, N) of the maze arm as spatial positioning references. In the training experiment, the platform was 2 cm below the water surface, and then the mice were put into the maze from the four water entry points. The mice were allowed to find the platform for 60 s, and the experiment was stopped when the mice stayed on the platform for 10 s. If the mice could not find the platform within 60 s, a ruler was used to guide the mice to the location of the platform and let the mice stay on the platform for 10 s. Each mouse was tested twice a day, entering the water from two different directions twice, and the interval between the two experiments for each mouse was at least 1 h. The escape latency of mice to find the platform, the total swimming distance of mice and the average swimming speed were recorded, and the learning and training were carried out for consecutive 7 days.

Platform experiment: On the 8^{th} day, the platform was removed, and then the mice were gently placed in the water from the diagonal position of the platform, and they were allowed to search the area where the original platform was located for 60 s. The number of times the mice shuttled through the area where the original platform was located (Number of crossing), and the swimming time periods of the mice in the target quadrant where the platform was located and the other three different quadrants (time in quadrants) were recorded. The water in the maze was changed every day, and the number and position of surrounding objects and experimenters remained unchanged.

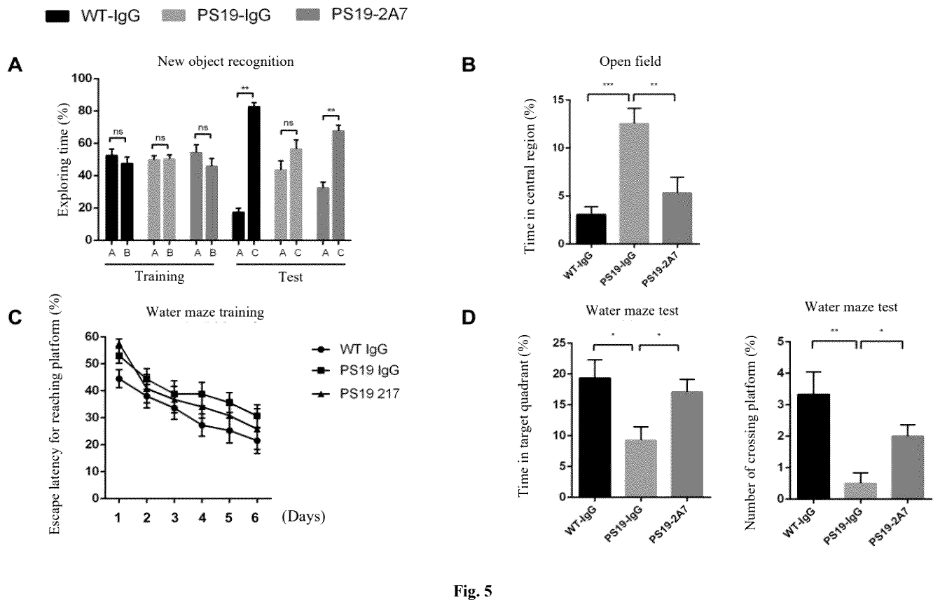

The experimental results were shown in FIG. 5. After treatment with 2A7 antibody, the behavior of 10.5-month-old PS19 mice was significantly improved. Specifically, in the new object recognition test, the time spent by the mice of the PS19-2A7 group exploring new object was significantly increased as compared with that of the PS19-IgG group (panel A in FIG. 5); in the open field test, after treatment with 2A7 antibody, the time spent by the PS19 mice in the central region of the box was significantly reduced, indicating that the mice's perception of surrounding light and spatial avoidance ability were improved (panel B in FIG. 5). The Morris water maze test showed that the spatial learning and memory ability of the mice in the PS19-2A7 group was significantly improved as compared with that of the PS19-IgG group (panels C and D in FIG. 5).

### EXAMPLE 6. NUCLEAR MAGNETIC RESONANCE DETECTION OF HIPPOCAMPAL VOLUME CHANGES IN PS19 MICE AFTER 2A7 ANTIBODY TREATMENT

After the behavioral study, the mice (10.5 months old) in the above experiment were subjected to magnetic resonance imaging (MRI). The hippocampal volume of PS19 mice that were not treated or treated with ineffective antibodies would be significantly atrophied, and the therapeutic effect of 2A7 antibody can be evaluated by the degree of atrophy of the hippocampal volume of mice.

### Acquisition and Analysis of Magnetic Resonance Imaging Data

All MRI experiments on experimental animal were performed on a 9.4 T Bruker small animal imager, and the coil used in the experiment was a small animal brain coil. During the animal imaging process, the mice were anesthetized with a 1.5% isoflurane/oxygen mixture, and the respiratory status of the mice was monitored in real time using a respiratory monitoring sensor. A scanner was used to perform imaging scanning of axial T1WI-3D anatomical structure and T2WI-3D pathological structure; the MRI scanning sequence and parameters were as follows: (1) T1-weighted MRI: TR (time of repetition)=2500 ms, TE (time of echo)=33 ms, field of view=2×2 cm, matrix=256×256, SI (slice interval)=0.5 mm, FA (flip angle)=180°, slices=15. The MRI scan results were analyzed using imageJ, and 3D reconstruction of the hippocampi was performed.

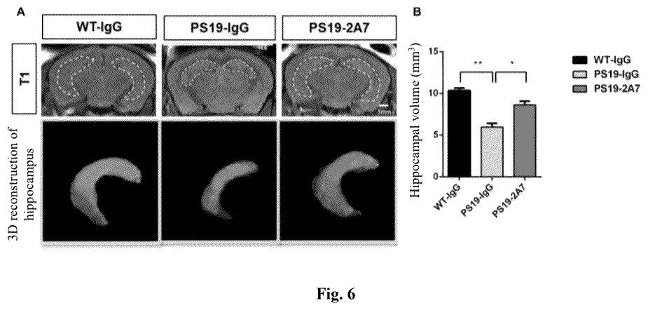

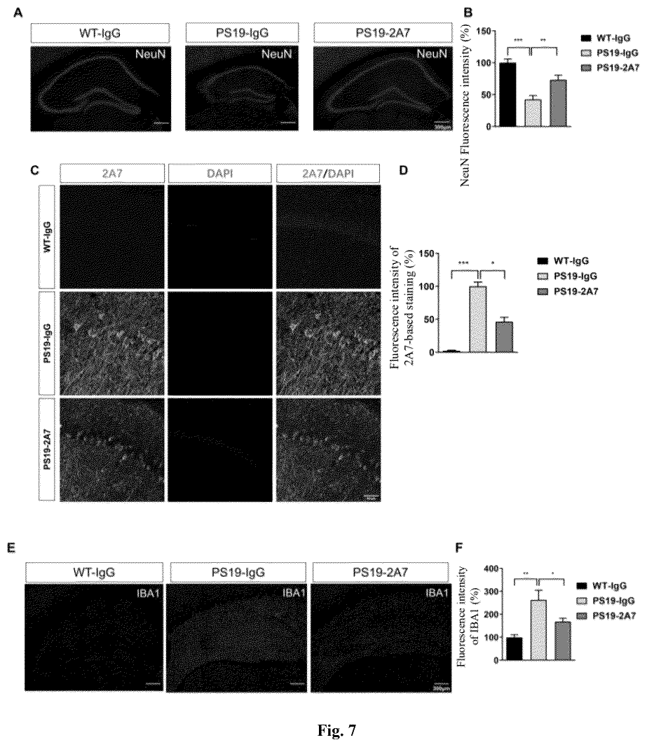

The experimental results were shown in FIG. 6. Therein, panel A showed the results of axial anatomical structure (T1) and 3D reconstruction of the brains of mice in the WT-IgG, PS19-IgG and PS19-2A7 groups detected by MRI; and panel B showed the statistical results of the hippocampal volume of mice in the WT-IgG, PS19-IgG and PS19-2A7 groups. The above results showed that after treatment with 2A7 antibody, hippocampal atrophy in 10.5-month-old PS19 mice was significantly inhibited.

### EXAMPLE 7. PATHOLOGICAL CHANGES IN BRAIN TISSUE OF PS19 MICE AFTER TREATMENT WITH 2A7 ANTIBODY

### 7.1 Fluorescent Antibodies and Usage Ratios

Antibodies: X=1:200; AT8=1:200 (Invitrogen, MN1020); IBA1=1:500 (Wako, 019-19741); NeuN=1:400 (CST, 24307)

### 7.2 Frozen Sections of Tissue

1. The mice in the above experiment were taken, anesthetized with 5% chloral hydrate, perfused with pre-cooled 1×PBS and 4% PFA (in 1×PBS, pH 7.4), and then the brains were peeled off, and continuously fixed in 4% PFA at 4° C. overnight;
2. On the second day, the fixative was decanted and replaced with 25% sucrose (in 1×PBS) at 4° C. for dehydration.
3. On the third day, the 25% sucrose was decanted and replaced with 30% sucrose (in 1×PBS) at 4° C. for dehydration. Sucrose was replaced daily so that 30% sucrose was continuously used for dehydration for 3 days. After the tissues were thoroughly dehydrated, the liquid on the surface of the brain tissue was sucked off with clean filter paper, and the brain was embedded with German Leica tissue freezing medium (JUNG tissue freezing medium), and then frozen sections were made at a thickness of 40 μm;
4. Intact and suitable sections were selected and placed in 1×PBS, rinsed at room temperature 3 times, 10 min for each time;
5. The PBS was discarded, blocking was performed with PBS containing 10% donkey serum (Solarbio, SL050) and 0.3% Triton X-100, and incubation was performed for 1 h at room temperature.
6. The blocking solution was pipetted and discarded, and incubation with primary antibody was performed; the primary antibody was diluted with blocking solution (containing 10% donkey serum+0.3% Triton X-100, in 1×PBS) to the desired concentration and incubated at 4° C. overnight;
7. The primary antibody was recovered, washing was performed with 1×PBST (1×PBS containing 0.3% Triton X-100) 3 times, 10 min for each time; the PBST was pipetted and discarded, and incubation with secondary antibody was performed; the fluorescent secondary antibody and DAPI were diluted with blocking solution at a ratio of 1:500 and 1:1000 respectively, added to the corresponding mouse brain tissue slices, and incubated at room temperature in the dark for 1 hour.
8. The secondary antibody was pipetted and discarded, washing was performed with 1×PBST 3 times, 10 min for each time; the PBST was pipetted and discarded, the brain slices were picked with a brush and placed in 1×PBS, fished with an adhesive slide, dried, and sealed with Solarbio anti-fluorescence quencher, and then the edges of coverslip were sealed well with nail polish, and storage was carried out at 4° C. in the dark.
9. An inverted laser confocal microscope (Zeiss 880) was used to take photos for analysis.

The experimental results were shown in FIG. 7, in which the immunofluorescence staining results in panels A and B showed that the neuronal loss in the mice of the PS19-2A7 group was significantly reduced; panels C and D showed that the phosphorylated p-tau 217 signal in the mice of the PS19-2A7 group was significantly reduced; panels E and F showed that the microglial proliferation in the mice of the PS19-2A7 group was significantly inhibited. The experimental results showed that after treatment with 2A7 antibody, the pathology of 10.5-month-old PS19 mice was significantly alleviated.

Although the specific embodiments of the invention have been described in detail, it will be understood by those skilled in the art that various modifications and variations to the details are possible in light of all the disclosed teachings and that these changes are within the scope of protection of the present invention. The entirety of the present invention is given by the appended claims and any equivalents thereof.

1. An antibody or antigen-binding fragment thereof capable of specifically binding to p-tau 217, wherein the antibody or antigen-binding fragment thereof comprises:
(a) a heavy chain variable region (VH) comprising the following 3 complementarity determining regions (CDRs):
(i) a VH CDR1, which consists of the following sequence: SEQ ID NO: 3, or a sequence having a substitution, deletion or addition of one or several amino acids (e.g., a substitution, deletion or addition of 1, 2 or 3 amino acids) as compared thereto,
(ii) a VH CDR2, which consists of the following sequence: SEQ ID NO: 4, or a sequence having a substitution, deletion or addition of one or several amino acids (e.g., a substitution, deletion or addition of 1, 2 or 3 amino acids) as compared thereto, and
(iii) a VH CDR3, which consists of the following sequence: SEQ ID NO: 5, or a sequence having a substitution, deletion or addition of one or several amino acids (e.g., a substitution, deletion or addition of 1, 2 or 3 amino acids) as compared thereto;
and/or,
(b) a light chain variable region (VL) comprising the following 3 complementary determining regions (CDRs):
(iv) a VL CDR1, which consists of the following sequence: SEQ ID NO: 6, or a sequence having a substitution, deletion or addition of one or several amino acids (e.g., a substitution, deletion or addition of 1, 2 or 3 amino acids) as compared thereto,
(v) a VL CDR2, which consists of the following sequence: SEQ ID NO: 7, or a sequence having a substitution, deletion or addition of one or several amino acids (e.g., a substitution, deletion or addition of 1, 2 or 3 amino acids) as compared thereto, and
(vi) a VL CDR3, which consists of the following sequence: SEQ ID NO: 8, or a sequence having a substitution, deletion or addition of one or several amino acids (e.g., a substitution, deletion or addition of 1, 2 or 3 amino acids) as compared thereto;
preferably, the substitution as described in any one of (i) to (vi) is a conservative substitution;
preferably, the CDR as described in any one of (i) to (vi) is defined according to the Kabat, Chothia or IMGT numbering system;
preferably, the CDR as described in any one of (i) to (vi) is defined according to the IMGT numbering system.

2. The antibody or antigen-binding fragment thereof according to claim 1, wherein the antibody or antigen-binding fragment thereof comprises:
(a) a heavy chain variable region (VH), which comprises an amino acid sequence selected from the group consisting of:
(i) a sequence as set forth in SEQ ID NO: 1;
(ii) a sequence having a substitution, deletion or addition of one or several amino acids (e.g., a substitution, deletion or addition of 1, 2, 3, 4 or 5 amino acids) compared to the sequence as set forth in SEQ ID NO: 1; or
(iii) a sequence having a sequence identity of at least 80%, at least 85%, at least 90%, at least 91%, at least 92%, at least 93%, at least 94%, at least 95%, at least 96%, at least 97%, at least 98%, at least 99%, or 100% as compared to the sequence as set forth in SEQ ID NO: 1;
and/or
(b) a light chain variable region (VL), which comprises an amino acid sequence selected from the group consisting of:
(iv) a sequence as set forth in SEQ ID NO: 2;
(v) a sequence having a substitution, deletion or addition of one or several amino acids (e.g., a substitution, deletion or addition of 1, 2, 3, 4 or 5 amino acids) compared to the sequence as set forth in SEQ ID NO: 2; or
(vi) a sequence having a sequence identity of at least 80%, at least 85%, at least 90%, at least 91%, at least 92%, at least 93%, at least 94%, at least 95%, at least 96%, at least 97%, at least 98%, at least 99%, or 100% as compared to the sequence as set forth in SEQ ID NO: 2;
preferably, the substitution described in (ii) or (v) is a conservative substitution.

3. The antibody or antigen-binding fragment thereof according to claim 1, wherein the antibody or antigen-binding fragment thereof comprises a constant region or variant thereof derived from a human immunoglobulin;
preferably, the antibody or antigen-binding fragment thereof comprises:
(a) a heavy chain constant region (CH) or variant thereof derived from a human immunoglobulin, wherein the variant has a substitution, deletion or addition of one or more amino acids or any combination thereof (e.g., a substitution, deletion or addition of up to 20, up to 15, up to 10, or up to 5 amino acids or any combination thereof, for example, a substitution, deletion or addition of 1, 2, 3, 4 or 5 amino acids or any combination thereof) as compared to the sequence from which it is derived; and/or
(b) a light chain constant region (CL) or variant thereof derived from a human immunoglobulin, wherein the variant has a substitution, deletion or addition of one or more amino acids or any combination thereof (e.g., a substitution, deletion or addition of up to 20, up to 15, up to 10, or up to 5 amino acids or any combination thereof, for example, a substitution, deletion or addition of 1, 2, 3, 4 or 5 amino acids or any combination thereof) as compared to the sequence from which it is derived;
preferably, the heavy chain constant region is an IgG heavy chain constant region, such as an IgG1, IgG2, IgG3 or IgG4 heavy chain constant region;
preferably, the light chain constant region is a κ light chain constant region.

4. The antibody or antigen-binding fragment thereof according to claim 1, wherein the antigen-binding fragment is selected from the group consisting of Fab, Fab′, F(ab′)², Fv, disulfide-linked Fv, BsFv, dsFv, (dsFv)², dsFv-dsFv′, scFv, scFv dimer, camelized single chain domain antibody, diabody, ds diabody, nanobody, single domain antibody (sdAb), bivalent domain antibody; and/or, the antibody is a mouse antibody, a chimeric antibody, a humanized antibody or a multispecific antibody.

5. The antibody or antigen-binding fragment thereof according to claim 1, wherein the antibody or antigen-binding fragment thereof carries a label; preferably, the antibody or antigen-binding fragment thereof carries a detectable label, such as an enzyme (e.g., horseradish peroxidase), a radionuclide, a fluorescent dye, a luminescent substance (e.g., a chemiluminescent substance) or biotin.

6. An isolated nucleic acid molecule, which encodes the antibody or antigen-binding fragment thereof according to claim 1, or a heavy chain variable region and/or a light chain variable region thereof;
preferably, the nucleic acid molecule comprises a nucleotide sequence as set forth in SEQ ID NO: 12 or SEQ ID NO: 13.

7. A vector, which comprises the nucleic acid molecule according to claim 6; preferably, the vector is a cloning vector or an expression vector.

8. A host cell, which comprises the nucleic acid molecule according to claim 6 or a vector comprising the nucleic acid molecule.

9. A method for preparing an antibody or antigen-binding fragment thereof, comprising culturing the host cell according to claim 8 under a condition that allows expression of the antibody or antigen-binding fragment thereof, and recovering the antibody or antigen-binding fragment thereof from a culture of the cultured host cell;
preferably, the host cell is a mammalian cell.

10. A multispecific molecule, which comprises the antibody or antigen-binding fragment thereof according to claim 1;
preferably, the multispecific molecule is capable of specifically binding to p-tau 217, and additionally specifically binding to one or more other targets;
preferably, the multispecific molecule further comprises at least one molecule (e.g., a second antibody or antigen-binding fragment thereof) having a binding specificity for a second target.

11. A pharmaceutical composition, which comprises the antibody or antigen-binding fragment thereof according to claim 1, or a multispecific molecule comprising the antibody or antigen-binding fragment thereof, and a pharmaceutically acceptable carrier and/or excipient;
preferably, the pharmaceutical composition further comprises an additional pharmaceutically active agent;
preferably, the additional pharmaceutically active agent is a drug having activity in treating tauopathy (e.g., AD).

12. A kit, which comprises the antibody or antigen-binding fragment thereof according to claim 1;
preferably, the kit is used to detect the presence or content of p-tau 217 in a sample;
preferably, the sample is a cerebrospinal fluid, whole blood, serum or plasma obtained from a subject;
preferably, the kit further comprises a reagent (e.g., horse serum) for diluting the sample.

13. A method for preventing and/or treating a tauopathy in a subject (e.g., a human), the method comprising administering to the subject in need thereof an effective amount of (i) the antibody or antigen-binding fragment thereof according to claim 1, or (ii) a multispecific molecule comprising the antibody or antigen-binding fragment thereof, or (iii) a pharmaceutical composition comprising the antibody or antigen-binding fragment thereof or the multispecific molecule and a pharmaceutically acceptable carrier and/or excipient;
preferably, the tauopathy is selected from the group consisting of Alzheimer's disease (AD), primary age-related tauopathy, chronic traumatic encephalopathy, Pick's disease and corticobasal degeneration;
preferably, the tauopathy is AD;
preferably, the medicament further comprises an additional pharmaceutically active agent having activity in treating tauopathy (e.g., AD);
preferably, the cerebrospinal fluid of the subject contains p-tau 217;
preferably, the subject is a mammal, such as a human;
preferably, the method further comprising administering an additional drug having the activity of preventing and/or treating AD.

14. A method for detecting the presence or amount of p-tau 217 in a sample, comprising the following steps:
(1) contacting the sample with the antibody or antigen-binding fragment thereof according to claim 1;
(2) detecting the formation of a complex of the antibody or antigen-binding fragment thereof and p-tau 217 or detecting the amount of the complex;
preferably, the antibody or antigen-binding fragment thereof carries a detectable label.

15. A method for detecting whether a subject suffers from a tauopathy, or for distinguishing a patient suffering from Alzheimer's disease (AD) from a patient suffering from other tauopathy, the method comprising:
detecting the amount of p-tau 217 in a sample by using the antibody or antigen-binding fragment thereof according to claim 1, and the sample is obtained from the subject or patient;
optionally, the method further comprises comparing the detected amounts of p-tau 217 in different samples to detect whether the subject suffers from Alzheimer's disease (AD), or to distinguish the patient with Alzheimer's disease (AD) from the patient with other tauopathy;
preferably, the method comprises:
(1) contacting the sample with the antibody or antigen-binding fragment thereof;
(2) detecting the formation of a complex of the antibody or antigen-binding fragment thereof and p-tau 217 or detecting the amount of the complex;
preferably, the tauopathy is selected from the group consisting of Alzheimer's disease (AD), primary age-related tauopathy, chronic traumatic encephalopathy, Pick's disease and corticobasal degeneration;
preferably, the tauopathy is AD;
preferably, the reagent detects whether a subject suffers from a tauopathy, or distinguishes a patient suffering from Alzheimer's disease (AD) from a patient suffering from other tauopathy by detecting the amount of p-tau 217 in a sample, and the sample is obtained from the subject or patient;
preferably, the sample is a blood sample (e.g., whole blood, serum, plasma) obtained from the subject or patient.

16. The antibody or antigen-binding fragment thereof according to claim 1, wherein the antibody or antigen-binding fragment thereof comprises the following three CDRs of a heavy chain variable region: a VH CDR1 as set forth in SEQ ID NO: 3, a VH CDR2 as set forth in SEQ ID NO: 4, a VH CDR3 as set forth in SEQ ID NO: 5; and/or, the following three CDRs of a light chain variable region: a VL CDR1 as set forth in SEQ ID NO: 6, a VL CDR2 as set forth in SEQ ID NO: 7, a VL CDR3 as set forth in SEQ ID NO: 8.

17. The antibody or antigen-binding fragment thereof according to claim 1, wherein the antibody or antigen-binding fragment thereof comprises: a VH having a sequence as set forth in SEQ ID NO: 1 and/or a VL having a sequence as set forth in SEQ ID NO: 2.

18. The antibody or antigen-binding fragment thereof according to claim 1, wherein the antibody or antigen-binding fragment thereof comprises a heavy chain framework region sequence and/or a light chain framework region sequence derived from a human immunoglobulin.

19. The kit according to claim 12, wherein the antibody or antigen-binding fragment thereof carries a detectable label, such as an enzyme (e.g., horseradish peroxidase), a radionuclide, a fluorescent dye, a luminescent substance (e.g., a chemiluminescent substance) or biotin.

20. The kit according to claim 12, wherein the kit further comprises a second antibody, which is capable of specifically recognizing the antibody or antigen-binding fragment thereof;
preferably, the second antibody further comprises a detectable label, such as an enzyme (e.g., horseradish peroxidase), a radionuclide, a fluorescent dye, a luminescent substance (e.g., a chemiluminescent substance) or biotin;
preferably, the second antibody is coated on magnetic beads.
